(19) United States
(12) Patent Application Publication
(10) Pub. No.: US 2013/0338145 A1
(43) Pub. Date: Dec. 19, 2013

(54) Method for Mediating Dopamine Receptor-Driven Reacidification of Lysosomal pH

(71) Applicants: Claire H. Mitchell, Philadelphia, PA (US); Alan M. Laties, Philadelphia, PA (US)

(72) Inventors: Claire H. Mitchell, Philadelphia, PA (US); Alan M. Laties, Philadelphia, PA (US)

(21) Appl. No.: 13/839,254

(22) Filed: Mar. 15, 2013

Related U.S. Application Data
(63) Continuation-in-part of application No. 12/418,328, filed on Apr. 3, 2009.
Application No. 12/418,328 is a continuation of application No. PCT/US2007/021211, filed on Oct. 3, 2007.
(60) Provisional application No. 60/849,050, filed on Oct. 3, 2006.
(60) Provisional application No. 60/966,086, filed on Aug. 23, 2007.

(51) Int. Cl. A61K 31/55; A61K 31/353

(52) U.S. Cl. CPC A61K 31/55; A61K 31/353
USPC 514/217.02; 514/456; 435/375

(57) ABSTRACT

Provided is a method of treating or preventing age-related macular degeneration (AMD) or Stargardt's disease in a patient subject to, or symptomatic of the disease, whereby normal lysosomal pH (pH_{L}) of compromised retinal pigment epithelium (RPE) cells of the eye is restored, or abnormally elevated pH_{L} is reacidified, thus decreasing or preventing damaging accumulations of lipofuscin debris or photoreceptor waste products. Further provided is a method for restoring photoreceptors to the eye of a patient subject to, or symptomatic of reduced photoreceptor activity or lipofuscin accumulation in RPE cells. By these methods D5 dopamine receptor (D5DR) agonists are administered to stimulate D5DR activity of compromised RPE cells, thereby regulating and reacidifying lysosomal pH (pH_{L}) by a D5 dopamine receptor-(D5DR)-mediated pathway, without altering baseline maintenance.

### CROSS-REFERENCE TO RELATED APPLICATIONS

This application is a Continuation-in-Part of U.S. application Ser. No. 12/418,328, published as US 2009/0247483 on Oct. 1, 2009, which is a Continuation of International Application PCT/US2007/021211 filed on Oct. 3, 2007 and published on Apr. 10, 2008, which claims priority to U.S. Provisional Application 60/849,050 filed on Oct.3, 2006 and U.S. Provisional Application 60/966,086 filed on Aug. 23, 2007, each of which is incorporated herein in its entirety.

### GOVERNMENT INTERREST

This invention was supported in part by funds from the U.S. Government (Department of Health and Human Services Grant Nos. EY-13434, EY-15537, EY-17045, and EY-018705) and U.S. Government may therfore have certain rights in the invvention.

### FIELD OF THE INVENTION

The invention relates to treatment of vision loss and retinal diseases, particularly macular degeneration, by modification of the pH of retinal pigment epithelial lysosomes, based upon manipulation of the lysosomal pH.

### BACKGROUND

Age-related macular degeneration (AMD) is the leading cause of untreatable vision loss in elderly Americans (Klein et al., Invest. Ophthalmol. Vis. Sci. 36:182-191 (1995)). The initial stages of the disease are neither well understood nor currently treatable. The photoreceptors of the retina comprise the rods and cones, each of which is a specialized sensory cell, a bipolar neuron. Each is composed of an inner and an outer region. The cone's outer segment, like that of adjacent rod photoreceptors, consists of a series of stacked cell membranes that are rich in photosensitive pigments. The distal tips of the rod outer segments are intimately associated with the outermost layer of the retina, the pigment epithelium (PE). The rod outer segments are in a continuous state of flux, wherein new stacks of membrane are added at the base of the outer segment, and old, worn-out stacks of membrane are shed from its distal tip. The shed rhodopsin-laden segments are phagocytosed by cells of the retinal pigment epithelium (RPE) and engulfed by lysosomes, becoming residual bodies in the cytoplasm of the epithelial cells. Daily phagocytosis of spent photoreceptor outer segments is a critical maintenance function performed by the RPE to preserve vision. Aging retinal pigment epithelium (RPE) accumulates lipofuscin, which includes N-retinylidene-N-retinylethanolamine (A2E) as the major autofluorescent component.

A2E is localized to lysosomes in cultured RPE, as well as in human RPE in situ. Thus, one of the earliest characteristics of the disorder is the accumulation of lipofuscin in the RPE (Feeney-Burns et al., Am. J Ophthalmol. 90:783-791 (1980); Feeney et al., Invest Ophthalmol Vis. Sci. 17:583-600 (1978)). A2E, a primary constituent of lipofuscin (Eldred et al., Nature. 361:724-726, 1993.)), undermines lysosomal organelles in several ways including by elevating lysosomal pH (pH_{L}) (Eldred et al., Gerontol. 2:15-28 (1995); Holz et al., Invest Ophthalmol Vis. Sci. 40:737-743 (1999)). As key lysosomal enzymes act optimally in a narrow range of acidic environments, an increase in pH_{L} reduces their degradative ability. Because of the circadian rhythm of RPE phagocytosis in the eye, a delay in lipid degradation results in a buildup of undigested material in RPE after 24 hours. A consequent accumulation of undigested material compromises RPE cells and appears to hasten the development of AMD. In this regard, the restoration of an optimal acidic environment to lysosomes could enhance enzyme activity and slow or stop the progression of AMD.

Dry AMD is characterized by the failure of multiple systems in the posterior eye and is associated with the accumulation of abnormal deposits within and upon Bruch's membrane (Moore et al., Invest Ophthalmol Vis. Sci. 36:1290-1297 (1995)), which separates the blood vessels of the choriod from the RPE layer. The RPE sends metabolic waste from the photoreceptors across Bruch's membrane to the choroid. The Bruch's membrane allows 2-way transit; in for nutrients and out for waste. Thus, Bruch's membrane's vital function is to supply the RPE and outer part of the sensory retina with all of their nutritional needs. However, as Bruch's membrane thickens and gets clogged with age, the transport of metabolites is decreased. This may lead to the formation of drusen, debris which can be seen in the eye as yellow-gray nodules located between the RPE and Bruch's membrane in age-related macular degeneration (Kliffen et al., Microsc Res Tech. 36:106-122 (1997); Cousins et al., In Macular Degeneration Eds. Penfold & Provis, Springer-Verlag, New York, pp. 167-200, (2005)). Drusen deposits vary in size and may exist in a variety of forms, from soft to calcified. With increased drusen formation the RPE are gradually thinned and begin to lose their functionality. While drusen formation is not necessarily the cause of dry AMD, it does provide evidence of an unhealthy RPE. There is also a buildup of debris deposits (Basal Linear Deposits or BLinD and Basal Laminar Deposits BLamD) on and within the membrane. Consequently, the retina, which depends on the RPE for its vitality, may be affected and vision problems arise.

While the initial triggers for these changes are not certain, decline in the hydraulic conductivity of Bruch's membrane, decreased choroidal perfusion (Lutty et al., Mol. Vis. 5:35 (1999)), environmental and immunologic injury (Beatty et al., Surv. Ophthalmol. 45:115-134 (2000); Zhang et al., J. Cell. Sci. 116:1915-1923 (2003)), genetic defects (Kuehn et al., J. Am. Med. Ass. 293:1841-1845 (2005); Ambati et al., Nature. Med. 9:1390-1397 (2003)), and other degenerative diseases (Johnson et al., Proc. Nat. Acad. Sci. USA 99:11830-11835 (2002); Mullins et al., FASEB. 1 14:835-846 (2000)) may all contribute to the development of the pathology. The identification of lysozyme C and oxidation products of docosahexaenoate in material present between Bruch's membrane and the RPE suggests that the extrusion of material from the lipofuscin-laden RPE contributes to sub-retinal deposit formation (Young et al., Surv. Ophthalmol. 31:291-306 (1987); Crabb et al., Proc. Nat. Acad. Sci. USA. 99: 14682-14687 (2002)). The correlation between RPE lipofuscin levels and those retinal regions showing the highest degree of atrophy supports the growing concept that lipofuscin is not just an indicator of disease, but rather, is itself a causal factor (von Ruckmann et al., Graefes Arch. Clin. Exp. Ophthalmol. 237:1-9 (1999); Roth et al., Graefes. Arch. Clin. Exp. Ophthalmol. 242:710-716 (2004)), suggesting that a reduction in the rate of lipofuscin formation and an enhancement in lysosomal degradative capacity will slow or stop the progression of AMD before substantial degeneration has occurred.

Lipofuscin in the RPE is primarily derived from incomplete digestion of phagocytosed photoreceptor outer segments (Young et al., Surv. Ophthalmol. 31:291-306 (1987); Eldred., In The Retinal Pigment Epithelium, Eds. Marmor & Wolfensberger, Oxford, University Press, New York, pp. 651-668, (1998)), with rates of formation reduced when photoreceptor activity is diminished (Katz et al., Exp. Eye. Res. 43:561-573 (1986); Sparrow et al., Exp. Eye. Res. 80:595-606 (2005)). A2E is a key component of RPE lipofuscin, with A2PE, iso-A2E and other related forms present (Eldred et al., supra, 1993; (Mata et al., Proc. Nat. Acad. Sci. USA 97:7154-7159 (2000)).

A2E has been identified in post-mortem eyes from elderly subjects, while levels are substantially elevated in Stargardt's disease, characterized by early-onset macular degeneration (Mata et al., supra, 2000). The disease is associated with mutations in the ABCA4 (ABCR) gene, whose product transports a phospholipid conjugate of all-trans-retinaldehyde out of the intradisk space of the photoreceptors (Allikmets et al., Nature. Gen. 15:236-246 (1997); Sun et al., Nature. Gen. 17:15-16 (1997)). The accumulation of substrate resulting from the transport failure leads to formation of A2PE, which is subsequently delivered to the RPE after the phagocytosis of the outer segments (Sun et al., J. Biol. Chem. 274:8269-8281 (1999)). A2PE is cleaved to A2E in the RPE, with small amounts of spontaneous isomerization to iso-A2E occurring (Parish et al., Proc. Nat. Acad. Sci. USA 95:14609-1413 (1998); Ben-Shabat et al., J. Biol. Chem. 277:7183-7190 (2002)). Measurements from ABCA4^{-/-} mice, developed by Travis and colleagues, have demonstrated that A2E levels are greatly enhanced in the RPE of ABCA4 mutant mice, consistent with the elevated levels of A2E in patients with Stargardt's disease (Mata et al., supra, 2000). In a rate-determining step in the visual cycle, retinaldehyde is reduced to retinol by the enzyme retinol dehydrogenase located in the photoreceptor outer segment. Thus, only the retinaldehyde that escapes conversion to retinol can react with phosphatidylethanolamine, and enter the A2E biosynthetic pathway to generate A2E in a multistep process.

The above-noted localization of A2E predominantly to lysosomes and late endosomes of RPE cells in vitro and in situ, is consistent with the phagolysosomal origins of lipofuscin granules (Holz et al., supra, 1999; Finnemann et al., Proc. Natl. Acad. Sci. USA 99:3842-3847 (2002)). As lysosomal organelles in the RPE degrade phagocytosed outer segments, the accumulation of undigested material of outer segment origin in AMD is consistent with a lysosomal dysfunction. Addition of A2E to cultured cells reduces the lysosomal degradation of photoreceptor outer segment lipids (Finnemann et al., supra, 2002), and decreases the pH-dependent protein degradation attributed to lysosomal enzymes (Holz et al., supra, 1999).

The mechanisms by which A2E causes lysosomal damage are influenced by levels of light and A2E itself. At high concentrations, the amphiphilic structure leads to a detergent-like insertion of A2E into the lipid bilayer, with consequent loss of membrane integrity and leakage of lysosomal enzymes (Eldred et al., supra, 1993; Sparrow et al., Invest. Ophthalmol. Vis. Sci. 40:2988-2995 (1999); Schutt et al., Graefes. Arch. Clin. Exp. Ophthalmol. 240:983-988 (2002)). Low-wavelength light can oxidize lipofuscin and A2E into toxic forms, which rapidly lead to cell death (Sparrow et al., supra, 2005; Sparrow et al., Invest. Ophthalmol. Vis. Sci. 41:1981-1989 (2000)). The direct effect on degradative lysosomal enzymes is also dependent on light. While lipofuscin directly decreases the activity of several lysosomal enzymes removed from lysosomes when exposed to light, it had little effect on their activity in the dark (Shamsi et al., Invest. Ophthalmol. Vis. Sci. 42:3041-3046 (2001)). The lack of direct effects on lysosomal enzymes in the absence of light treatment has been confirmed by Bermann et al., Exp Eye Res. 72:191-195 (2001).

Conversely, however, indirect effects are likely, since A2E interferes with the function of the lysosomal vH⁺ATPase proton pump (Bergmann et al., FASEB. J. 18:562-564 (2004)), and low levels of A2E increased lysosomal pH (Holz et al., supra, 1999). The detected lysosomal pH change indicated that A2E could reduce enzyme effectiveness by alkalizing the lysosomes. Yet, because this pH-dependent effect occurred at low levels of A2E that had little effect on membrane leakage, the alkalinization apparently preceded acute disruption of membrane integrity.

The modification and degradation of material by lysosomes is essential for cellular function. Lysosomal enzymes function optimally over a narrow range of acidic pH values and the predominant lysosomal enzymes of the RPE reflect this tight pH dependence. Lysosomes are characterized by their low pH (4.5-5.0), with optimal enzyme activity dependent on vesicle pH (Geisow et al., Exp. Cell. Res. 150:36-46 (1984)). Reported optimal lysosomal pH ranges (“normal pH_{L}”) are 4.0-5.0 (Hayaseet al., J. Biol. Chem. 245:169-175 (1970); Mego, Biochem. J. 218:775-783 (1984)). However, activity of the major RPE enzyme lysosomal acid lipase decreases by 60% when the pH is raised from 4.5 to 5.2, while activity of major protease cathepsin D falls by 80% when the pH rises from 4.5 to 5.0 [Hayasaka et al., supra, 1975; Barrett In Protinases in Mammalian Cells and Tissues, Elsiver/North-Hollard, Biomedical. Press, New York, pp. 220-224 (1977)). This sharp pH dependence of enzyme activity implied that alkalizing lysosomes of RPE cells will lower the activity of multiple enzymes and interfere with the degradation of internalized outer segments.

Elevation of cytoplasmic cAMP has been determined to restore the pH_{L} of compromised RPE cells to more acidic levels (Liu et al., Invest Opthamol. Vis. Sci. 49:772-780 (2008)). The degradation of outer segments of the photoreceptor is primarily mediated by the aspartic protease cathepsin D (Hayasaka et al., J. Biochem. 78:1365-1367 (1975)). While its pK_{A} varies with substrate, the degradative activity of cathepsin D is generally optimum near pH 4, and falls below 20% of maximum at pH>5.0 (Barrett, supra, 1977). Rats treated with chloroquine, which is known to alkalize lysosomes (Krogstad et al., Am. J Trop. Med. Hyg. 36:213-220 (1987)), doubled the number of outer segment-derived lysosome-associated organelles in the RPE (Mahon et al., Curr. Eye. Res. 28:277-284 (2004)), leading to the finding that lysosomal alkalization by A2E contributes to the accumulation of lipofuscin in the AMD. Conversely, epinephrine, norepinephrine and beta adrenergic agonist isoproterenol reacidified lysosomes; while the alpha adrenergic receptor agonist phenylephrine had no effect, and beta receptor antagonist timolol blocked the reacidification induced by norepinephrine (Liu et al., supra, 2008). However, pharmacologic restoration in a disorder that progresses over decades can be fully realized only when the mechanisms controlling lysosomal pH are understood.

Thus, a need has remained in the art, until the present invention, to find better ways to slow the progression of AMD, particularly by regulating the acidity of the lysosomes within the RPE cells.

### SUMMARY OF THE INVENTION

The present invention provides a method for slowing the progression of AMD by restoring an optimal acidic pH to compromised lysosomes in the RPE, and identifies compounds that lower lysosomal pH and increases the activity of degradative enzymes. By combining a mechanistic analysis of lysosomal acidification with a high through-put evaluation of the pharmacologic approach and the application of these findings to animal models, the present invention has determined methods for regulating lysosomal pH (pH_{L}) in the RPE cells.

It is, therefore, an object of the invention to provide methods of pharmacologic manipulation to treat, prevent and/or restore a perturbed lysosomal pH and enhance degradative ability in RPE cells. The absolute value over which the defect occurs in the RCE cells of ABCA4^{-/-} mice (animal model of AMD) is highly relevant to the determination of how to change pH_{L} and how to quantify that change, particularly as applied in humans.

It is a further object to determine the role of D1- and D5-like dopamine receptors and their corresponding receptor agonists in the chain of events resulting in the lowering of OI_{L} in RPE cells. This effect is measured in both cultured RPE cells, and in the actual defective RCE cells from ABCA4^{-/-} and bovine model animals. Thus, an effective treatment is provided by the present invention for reversing the abnormally elevated pH_{L} associated with macular degeneration, particularly for the macular degeneration found in AMD and in Stargardt's disease, and for restoring the damage caused by the increased pH_{L} in the patient's eye.

It is yet another object of the invention to offer distinctions between the effect of the D1DR and the D5DR on the reduction of lysosomal pH_{L} in compromised or alkalized RPE cells; and also to demonstrate a clear link between stimulation of the D5 receptor, reduction of lysosomal pH, and improved degradation by lysosomal enzymes.

Additional objects, advantages and novel features of the invention will be set forth in part in the description, examples and figures which follow, all of which are intended to be for illustrative purposes only, and not intended in any way to limit the invention, and in part will become apparent to those skilled in the art on examination of the following, or may be learned by practice of the invention.

### BRIEF DESCRIPTION OF THE FIGURES

The foregoing summary, as well as the following detailed description of the invention, will be better understood when read in conjunction with the appended drawings. It should be understood, however, that the invention is not limited to the precise arrangements and instrumentalities shown.

FIG. 1 diagrammatically presents an embodiment of the invention showing lysosomal vesicular acidification.

FIGS. 2A-2D are graphs showing elevation of pH_{L} and outer segment degradation by ARPE-19 cells. FIG. 2A shows that A2E (14 nM)±LDL elevated pH_{L}, but LDL itself had an effect. pH is normalized to the mean control of each week (n=8). FIG. 2B shows that incubation with tamoxifen (Tmx) raised pH_{L}. Symbols are mean±SEM fit with a single exponential curve (all n=30, all diff from 0 mM, p<0.001). FIG. 2C shows that the effect of tamoxifen was neither mimicked nor inhibited by 17-β-estradiol (17-β, n=6). FIG. 2D shows that tamoxifen and chloroquine (CHQ) slowed clearance of outer segments labeled with calcein after 24 hrs. n=12 for all.

FIGS. 3A-3D are graphs showing the effect of adrenoceptor agonists and cAMP lower pHL in ARPE-19 cells. FIG. 3A shows that adrenoceptor agonists norepinephrine (Nor) and epinephrine (Epi) and isoproterenol (Iso) helped restore pHL raised by tamoxifen (n=20-45). FIG. 5B shows that the acidification by norepinephrine was blocked by the β-adrenoceptor inhibitor, timolol (Tim, n=8). FIG. 3C shows that norepinephrine also acidified cells exposed to chloroquine (CHQ, n=20). FIG. 3D shows that cell permeant cAMP analog cpt-cAMP acidified the cells exposed to 10 and 30 μM tamoxifen (n=22-88).

FIG. 4 is a bar graph showing that ABCA4^{-/-} mice had an increased ratio of dye at 340/380 nm, consistent with an increased lysosomal pH, and consistent with the elevation found when A2E was added to ARPE-19 cells, showing that elevated pH occurs in an animal model of Stargardt's disease.

FIGS. 5A-5D are graphs showing the degree to which lysosomal pH is altered in ABCA4^{-/-} mice, and restoration of lysosomal pH with D1-like dopamine receptor agonists. FIG. 5A shows that pH_{L} was increased in RPE cells from ABCA4^{-/-} mice (n=6 trials, from 26 mice aged 216±28 days) compared to cells from wild type mice (n=7 trials, from 22 mice aged 215±32 days). FIG. 5B shows that lysosomal pH increases with the age of ABCA4^{-/-} mice (n=4, 2 mice each, MO=months old). FIG. 5C shows that dopamine D1-like receptor agonists A68930 and A77636 decreased lysosomal pH of ARPE-19 cells treated by tamoxifen (n=8). FIG. 5D shows that dopamine D1-like receptor agonists A68930 and A77636 decreased pH_{L} of RPE cells from 11-month-old ABCA4^{-/-} mice (n=8). In FIG. 5D, values are given as the ratio of light excited at 340 to 380 nm, an index of lysosomal pH.*=p<0.05, **=p<0.01, ***=p<0.001 vs control. Bars=mean±SEM.

FIGS. 6A-6D are graphs showing that D1-like receptor agonists lower lysosomal pH (pH_{L}) in challenged ARPE-19. FIG. 6A shows that the agonist A68930 acidified pH_{L} to 5.0 or lower in ARPE-19 cells challenged by tamoxifen (TMX) (n=14-40). FIG. 6B shows that the D1-like agonist A77636 also reduced pH_{L} in cells exposed to TMX (n=44). FIG. 6C shows that the D1-like agonist SKF 81297 also acidified the lysosomes of cells treated with TMX (n=20). FIG. 6D shows that the myristolated protein kinase inhibitor (14-22) amide, the cell-permeant inhibitor of protein kinase A, blocked the acidifying effects of SKF 81297 on cells treated with TMX, implying a role for protein kinase A in restoring pH_{L} (n=94) (#p<0.05 vs. control; *p<0.05 vs. TMX; **p<0.05 vs. SKF 81297).

FIGS. 7A-7B show the long-term restoration of pH_{L}. FIG. 7A shows D1-like agonist SKF 81297 restored pH_{L} for up to at least 12 days in compromised ARPE-19 cells. # CHQ versus control, p<0.05, *p<0.05 SKF 81297 versus CHQ; n=16-40. FIG. 7B shows the relative effectiveness of SKF 81297 expressed as a percentage of the control pH in the same plate on the same day.

FIGS. 8A-8C show that the simulation of the D5 receptor restores lysosomal acidity. FIG. 8A is a series of Western blots confirming specificity of the gene knockdown, as siRNA against the D1 receptor reduced expression of the D1 receptor (D1DR), but not the D5 receptor (D5DR, top panel). FIG. 8B shows that RNAi knockdown of D5 receptor - but not D1 receptor—reduced acidification by 10 μM D1/D5 agonist SKF 81297. TransCon=transfection control. Scr=scrambled RNAi. D1RNAi=RNA against D1 receptor. D5RNAi=RNA against D5 receptor. FIG. 8C shows the quantification of effect of receptor knockdown.

FIGS. 9A-9D show that D5 agonists reduce levels of photoreceptor outer segment auto-fluorescence. FIG. 9A (images i-vi) shows cultured ARPE-19 cells examined by confocal fluorescence microscopy following 7 days of incubation without A(i) or with A(ii) unlabeled photoreceptor outer segments (POS). Lipofuscin-like cellular autofluorescence was detected in A(ii) using a fluorescein filter set (ex 480 nm, em 535 nm). Nuclei were visualized by DAPI staining. Scale bar=10 μM. Autofluorescence associated with POS incubation A(iii) and the signal from LysoTrackerRed (ex 540 nm, em>570) showed considerable overlap A(vi) implying the majority of POS were in acidic organelles 2 h after outer segments were removed from the bath, A(v) DIC image. Scale bar=10 μM. FIG. 9B shows SKF 81297 reduced the autofluorescence from internalized POS. FIG. 9C shows quantification of autofluorescence reduction by SKF 81297. FIG. 9D shows Bodipy-pepstatin A binding is improved by addition of SKF 81297.

FIGS. 10A-10B show acidification of retinal pigmented epithelial (RPE) lysosomes from ABCA4^{-/-} mice. FIG. 10A shows simulation of dopamine D1-like receptors by D5DR agonists, A68930 (1 μM) and A77636 (1 μM) decreased pH_{L} of RPE cells freshly isolated from 11-month-old ABCA4^{-/-} mice. *p<0.01 versus untreated ABCA4^{-/-}. n=8 measurements. FIG. 10B shows that in a separate set of experiments, SKF 81297 (50 μM) also reduced the lysosomal pH in RPE cells freshly isolated from 12-month-old ABCA4^{-/-} mice. *p<0.05 versus untreated ABCA4^{-/-}. n=3.

FIGS. 11A-11C show measurement of intracellular calcium with the indicator fura-2 confirmed that raising lysosomal pH (increasing alkalization) with chloroquine led to the release of Ca2+ into the cells. Howver, this chloroquine-dependent release of calcium was attenuated by administering 10 μM SKF 81297 (n=12). Similarly, raising lysosomal pH with bafilomycin or tamoxifen caused a release of cytokine IL-6 into the extracellular bath (n=9). *p<0.05, which was also attenuated by administration of a D5DR agonist (SKF 81297).

### DETAILED DESCRIPTION OF CERTAIN EMBODIMENTS OF THE INVENTION

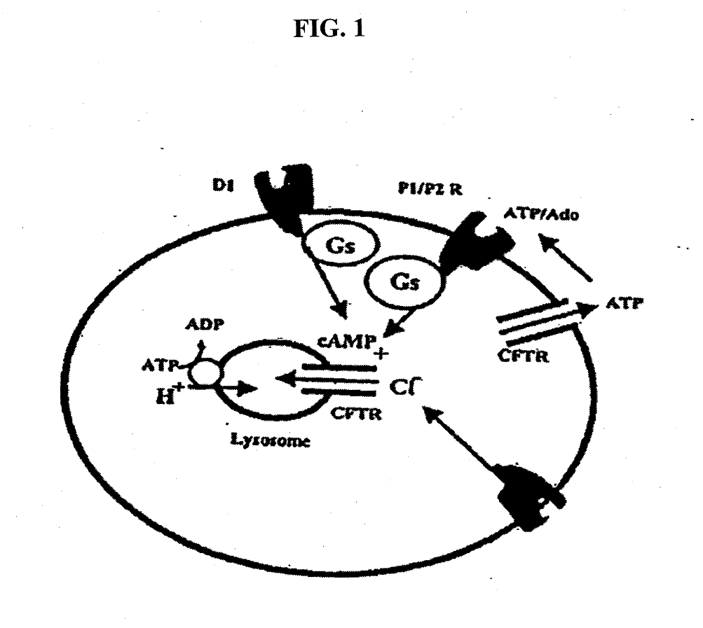

Changes in lysosomal pH have direct and indirect actions on activity of degradative lysosomal enzymes. FIG. 1 summarizes the invention as embodied when the lysosomal pH (pH_{L}) is restored following alkalization by A2E, e.g., as in the early stages of macular degeneration. Restoration increases activity of degradative enzymes and slows the rate of lipofuscin accumulation. Thus, the present invention provides methods, whereby as demonstrated in RPE cells, stimulation of the D5 dopamine receptor enhances degradation and increases beneficial activity of the degradative lysosomal enzymes under conditions wherein cells have been “compromised,” meaning that lysosomal pH has increased to an abnormal level, resulting from cellular aging of the photoreceptor debris clearance, or deterioration, or as induced by exposure to a dopamine receptor activity modifying protein, e.g., chemically induced by exposure to tamoxifen or chloroquine.

Dopamine receptors are a class of metabotropic G protein-coupled receptors that are prominent in the vertebrate central nervous system (CNS). The neurotransmitter dopamine is the primary endogenous ligand for dopamine receptors. These receptors have key roles in many processes, including the control of normal motor function and learning, as well as modulation of neuroendocrine signaling. There are five subtypes of dopamine receptors, D1, D2, D3, D4, and D5. D1 and D5 receptors share over 80% homology (Beaulieu and Gainetdinov, Pharmacol. Rev. 63:182-217 (2011)) and are members of the “D1-like family of dopamine receptors,” or “D1DR,” whereas the D2, D3 and D4 receptors are members of the “D2-like family.” For the purposes of this invention, D1-like receptors are defined as a subset, the “D1 (D1α) dopamine receptor” or “D1DR;” or as “D5 (D1β) dopamine receptors” are also referred to as “D5DR.” Both subtypes “D1/D5” receptors are stimulated or enhanced by exposure to “D1-like receptor agonists” and antagonists. See U.S. Pat. No. 6,469,141 and the references cited therein, wherein calcyon is defined as a D1 dopamine receptor activity modifying protein.

Activation of the D1-like family receptors is coupled to the G protein Gas, which subsequently activates adenylyl cyclase, increasing the intracellular concentration of the second messenger, cyclic adenosine monophosphate (cAMP). Increased cAMP in neurons is typically excitatory and can induce an action potential by modulating the activity of ion channels. A specific D1-like receptor agonist, A77636, reduces Parkinsonian activity in a primate model of the disease when delivered orally (Smith et al., J. Neur. Trans. 109:123-140 (2002).). Chronic administration of D1-like receptor agonists has also been used as a long-term treatment for Parkinson's disease, demonstrating the relative safety of long-term use of the drug in humans (Lewis et al., CNS & Neurol. Disord. Drug Targets 5:345-353 (2006); Mailman et al., Curr. Op. Invest. Drugs 2:1582-1591 (2001)). Abnormal dopamine receptor signaling and dopaminergic nerve function is implicated in several neuropsychiatric disorders. Most known side effects of A77636 are tolerable, or even beneficial, including increased cognitive ability (Stuchlik et al., Behay. Br. Res. 172:250-255 (2006)) and improved memory (Cai et al., J. Pharm. Exp. Ther. 283:183-189 (1997)).

While the identification of compounds that can acidify defective lysosomes has direct implications for the health of RPE cells, the development of optimal treatments requires an understanding of the mechanisms controlling pH_{L}. Previous work has investigated the role of dopamine receptors in the regulation of pH_{L}. However, while studies have indicated that the agonists of the D1-like family of receptors play can lower pH_{L}, there was a lack of clarity as to which specific receptor, D1 or D5, governed the reacidification of pH_{L}. Further, D1-like receptor agonists, such as A77636, have been shown to act on both D1 and D2 receptors. But because D2 receptors are coupled to Gi proteins, stimulation would work negatively, against an acidification.

Three different D1-like receptor agonists, A68930, A77636, and SKF 81297, all were tested and reacidified compromised lysosomes in RPE cells, demonstrating the effect of the class of compositions. Such reacidification occurred in lysosomes alkalized by either tamoxifen or chloroquine. The acidification was dependent on the actions of PKA, consistent with pathways identified previously (Liu et al. 2008). Of note, a single dose of agonist SKF 81297 was sufficient to acidify lysosomes for at least 12 days, with complete restoration found maximally 5-7 days after treatment. Knockdown of the D5 receptor reduced the acidification by SKF 81297, whereas knockdown of the D1 receptor did not, implying that the D5 receptor was responsible.

Embodiments of the invention have identified the differing extents to which D1 and D5 receptors affect pH_{L}, and have further identified, e.g., that SKF 81297 specifically increased the degradation of photoreceptor outer segments and reduced their lipofuscin, like autofluorescence and the activity of cathepsin D, supporting a link between lysosomal acidification and increased activity of degradative enzymes. Finally, stimulation of the receptor lowered lysosomal pH of RPE cells from aged ABCA4^{-/-} mice, demonstrating that the pathways linking the D5 receptor to lysosomal acidification were maintained—even in compromised RPE cells from “middle aged” mice. Overall, these findings demonstrate a clear link between stimulation of the D5 receptor, reduction of lysosomal pH, and improved degradation by lysosomal enzymes. Moreover, the control of lysosomal function in supportive cells may also have broader implications for neuronal-glial interactions. Recently, astrocytes were shown to actively phagocytose material extruded from the midst of axons (Nguyen et al. Proc. Natl. Acad. Sci. USA 108:1176-1181 (2011)). It remains to be seen whether alkalinization of astrocytic lysosomes can impede this novel function, or whether stimulation of the D5DR can enhance this process in axons.

Embodiments of the invention focus on the absolute values of the abnormally elevated pHL in the defective lysosomes in the RPE cells of a patient with AMD or Stargardt's disease, thus permitting correction of the pH to normal levels, restoring the damage associated with macular degeneration. Further, specific drugs are identified in this invention by combining a mechanistic analysis of lysosomal acidification with a high through-put evaluation of this pharmacologic approach. Thus, methods are provided in the present invention for slowing the progression of macular degeneration, specifically AMD and Stargardt's macular degeneration, by restoring an optimal acidic pH to compromised lysosomes in the RPE of the patient's eye.

Receptor pharmacology: Analysis of individual dopamine receptors is complicated by the lack of specificity demonstrated by many of the pharmacological tools. For example, as noted above, D1 and D5 (D1b) receptors share over 80% homology (Beaulieu and Gainetdinov, supra, 2011). Selective reduction of the D1 and D5 receptors using molecular approaches demonstrated that the acidification of lysosomes in RPE cells was mediated by D5 receptors. Although cultured bovine RPE cells were reported to contain predominantly D5 receptors (Versaux-Botteri et al., Neurosci. Letts. 237:9-12 (1997)), the presence of bands in the present study suggests cultured human ARPE-19 cells contain both D1 and D5 receptors. Although receptor expression may be coordinated, it is clear from the results provided by this present invention that the D5 receptor mediated lysosomal reacidification in these cells.

The agonists A77636 and A68930 are generally characterized as D1-like receptor agonists, but within that family, they are molecularly D5DR agonists. A68930 acts at D1 receptors with an EC₅₀ of 2.9 nM, and at D2 receptors with an EC₅₀ of 3.8 μM (DeNinno et al. Eur. J. Pharmacol. 199:209-219 (1991)). A77636 acts at D1-like receptors with a Ki=39.8 nM and at D2-like receptors with a Ki>101M, however (Kebabian et al., Eur. J. Pharmacol. 229:203-209 (1992)). Enhanced stimulation of D2 receptors at higher concentrations may complicate effects of A68930 on lysosomal acidification; as D2 receptors are coupled to Gi proteins stimulation would work against an acidification. However, the relative selectivity of A68930 at the D1 versus D5 receptor may also contribute to the response (Nerg\dot{a}rdh et al. Pharmacol. Biochem. Behay. 82:495-505 (2005)). Although SKF 81297 is reported to act more selectively at D1 receptors (Beaulieu and Gainetdinov, supra, 2011), present results argue that it is also an effective agonist at D5 receptors. It should be noted that although the majority of experiments in this study were performed with SKF 81297, this does not rule out possible beneficial effects from other D1/D5 agonists. The oral availability of A77636 may be of interest in this regard (Kebabian et al., supra, 1992). Other known D1/D5 dopamine receptor agonists (D5DR agonists), including the exemplified SKF 81297 composition, are available from Sigma-Aldrich (sigmaaldrich.com) St. Louis, Mo. See following list of D1/D5 dopamine receptor agonists (D5DR agonists):

| | |
|---|---|
| A68930 (hydrochloride) | Dinoxyline |
| A77636 | Doxanthrine |
| A86929 | Fenoldopam |
| 6-Br-APB | Pergolide |
| Cabergoline | SCH 23390 |
| CY 208243 | SKF 38393 (hydrochloride) |
| 7,8-dihydroxy-5-phenyl-octahydrobenzo | SKF 82958 |
| [h]isoquinoline | |
| dinapsoline | SKF 83822 (hydrobromide) |
| SKF 89145 | SKF 83959 (hydrobromide) |
| SKF 89626 | SKF 81297 (hydrobromide) |

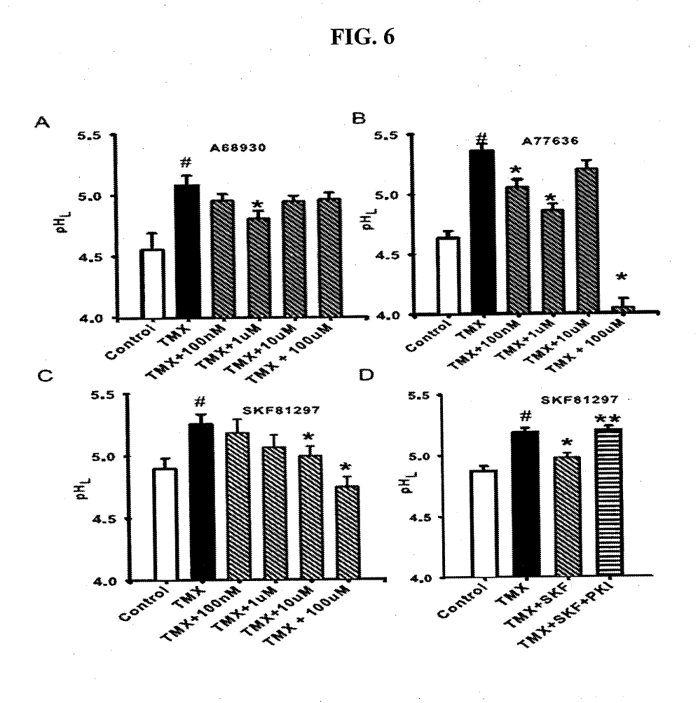

Mechanisms of action: The ability of D5 receptor stimulation to lower lysosomal pH is most likely related to an elevation of cAMP levels. It has been previously demonstrated that increasing cytoplasmic cAMP, either directly or via G-protein-coupled receptors, lowers lysosomal pH in RPE cells (Liu et al., supra, 2008). FIG. 6D demonstrates that the acidifying actions of the agonist SKF 81297 are inhibited by PKI (14-22) amide, strongly suggesting that PKA is required for lysosomal acidification. Preliminary data has indicated that the PKA-activated Cl⁻ channel CFTR (cystic fibrosis transmembrane conductance regulator channel/contributes to the PKA-dependent acidification of RPE lysosomes (Mitchell et al., Am. J. Physiol. Cell. Physiol. 276:C659-C666 (2008)). Also, phosphorylation by PKA was recently demonstrated to enhance insertion of the vHATPase into the plasma membrane of proton-secreting kidney cells, enhancing secretion (Alzamora et al,. J Biol. Chem. 285:24676-24685 (2010)). The inability of SKF 81279 to decrease baseline lysosomal pH is consistent with data indicating cAMP exhorts an acidification of greater magnitude from cells with alkalized lysosomes than from baseline (Liu et al., Amer. J. Physiol.—Cell Physiol. 303(2):C160-169 (July 2012)). This provides a model where the cAMP increase following D5DR stimulation affects the regulation of lysosomal pH, but not its baseline maintenance.

It is important to note that stimulation of the D5 receptor effectively reacidified RPE cells, overriding the alkalinization caused by either tamoxifen or chloroquine. Further, receptor stimulation reacidified RPE cells from ABCA4)/) mice, where excess A2E is likely to increase lysosomal pH (Holz et al., supra, 1999; Mata et al., supra, 2000; Bergmann et al., supra, 2004). Overall, this implies that the ability of D5 receptor stimulation to reacidify lysosomes is not specific for a particular type of alkalizing insult. In other words, the lysosomal reacidification is mediated via a general mechanism that may be effective against a range of insults.

Physiological Implications: Stimulation of the D5 receptor in RPE cells by a D5DR agonist, such as SKF 81297, induced several responses confirming the significance of further consideration. A single exposure to 10 μM SKF 81297 lowered lysosomal pH in chloroquine-treated ARPE-19 cells for at least 12 days. The tests ended at that point because 12 days generally is the maximum period for which cultured ARPE-19 cells can usually be viably maintained. The restoration of acidity was cumulative, with the pH equal to control levels after 7 days. Importantly, the autofluorescence excited at 488 nm was substantially increased in cells fed outer segments, consistent with a lipofuscin-like accumulation. However, treatment with SKF 81297 decreased this autofluorescence by 54±4%. Not only does the improved clearance by SKF 81297 reinforce the relationship between lysosomal pH and degradative enzyme activity, but it also provides crucial functional evidence that this approach can improve the clearance of outer segments by these cells.

It is important to stress that the pulse-chase approach to feeding cells outer segments ensured that outer segments were predominantly within lysosomes before cells were treated with SKF 81297, implying the actions were specifically due to changes in lysosomal pH and not the binding or internalization stages. As such, the approach also applies to material delivered through autophagic pathways to the lysosomes. Experiments with Bodipypepstatin provide additional support for this link, and stress that the reacidification induced by SKF-81297 occurs over a relevant pH values. Like many lysosomal enzymes, the activity of Cathepsin D is sharply dependent of the pH of the surrounding milieu, with activity falling by 80% once the pH has risen to only 5.3 (Barrett, supra, 1977). These experiments demonstrate that the functional effects of SKF-81297 on compromised RPE cells are substantial and demonstrate an improved degradation of compromised lysosomes in RPE cells.

The ability of D5 receptor stimulation to enhance outer segment degradation in RPE cells with alkalized lysosomes have implications for patients with macular degenerations, such as Stargardt's disease, for the lysosomal pH was increased in RPE cells from the ABCA4)/) mouse model of the disease (Liu et al. supra, 2008). As such, the ability of receptor agonists to acidify lysosomes from RPE cells taken from older ABCA4)/) mice is important, for it implies that the mechanisms necessary to mediate receptor-driven reacidification of lysosomes are still functioning, even though the lysosomes in the cells have been distressed for an extended period. The lysosomal pH increased with age in these mice (Liu et al., supra, 2008), consistent with the enhanced accumulation of A2E with age (Mata et al., supra, 2000). The negligible effect of DSDR agonists in younger mice with near-normal lysosomal pH may be related to the increased magnitude of acidification induced by cAMP when given to cells with alkalized lysosomes. This is also supported by the observation that SFK 81297 had no effect on cells that had not been treated with an alkalizing agent. This makes the treatment of impaired tissue with D5 agonists ideally suited, as the lysosomal pH of any healthy cells should be minimally affected.

Measuring Lysosomal pH in RPE Cells: In an embodiment of the invention, drugs were identified that lowered lysosomal pH (pH_{L}), recognizing the importance of acidic lysosomal pH for the degradative functions of the RPE and that pH_{L} may be elevated by A2E in early AMD. This required the development of an efficient protocol to screen pH_{L}. Traditional dyes have used fluorescence intensity as an index of pH. However, the ratiometric qualities of Lysosensor Yellow/Blue fluoresced yellow, making readings possible that are independent of dye concentration, providing a clear advantage in acidic organelles, like lysosomes, where the volume fluctuates with the pH (Pothos et al., J. Physiol. 542:453-476 (2002); Li et al., Am. J. Physiol. Cell. Physiol. 282:C1483-C1491 (2002)).

ARPE19 is a spontaneous, immortalized human RPE cell line obtained initially from a single human donor, now available at ATCC. Due to its immortality, this cell line has been studied extensively over the last decade to obtain important insights into RPE cell biology. See, e.g., Dunn et al., Exp. Eye Res. 62:155-69 (1996)). As a result, experiments in ARPE-19 cells were used to verify the source of the signal from Lysosensor Yellow/Blue and to optimize recording conditions.

Lysosensor Yellow/Blue co-localized with the Lysotracker Red dye in small vesicles, with a distribution consistent with lysosomal origin. Measurements of pH_{L} were performed using a high throughput screening (HTS) protocol to maximize output and minimize variation using ARPE-19 cells in 96 well plates. HTS assays are particularly useful in the present invention because of the ability to screen hundreds, thousands, and even millions of compounds in a short period of time. Loading for 5 min. at 23° C. with 5 μM Lysosensor, followed by 15 min. for internalization, produced stable and reproducible results.

The ratio of fluorescence (em >527 nm), typically excited at 340 nm and 380 nm, was measured for 20 msec, every 30 seconds, to minimize bleaching, and to determine the response to NH₄Cl. The ratio was converted to pH by calibrating with KCl buffered to pH 4.0-6.0 in the presence of monensin and nigericin. Calibration indicated a baseline pH of 4.4 to 4.5, supporting lysosomal localization. NH₄Cl (10 mM) increased fluorescence excited at 340 nm, increasing ratios (pH was elevated by 10 mM NH₄Cl (n=20, p<0.0001)), by the vH⁺ATPase inhibitor bafilomycin-A (pH was elevated by 200 nM BAF (n=20, p<0.0001)) and by chloroquine (pH was elevated by 20 μM CHQ (n=20, p<0.0001)), as expected. NH₄Cl decreased the ratios slightly at 380 nm. Nevertheless, absent the addition of the dye, none of these compounds, or any others, altered the fluorescent signal at 340 or 380 nm, showing a specificity of the measured change to pH_{L}. Thus, these results validate the use of the Lysosensor probe to measure pH_{L} using high through-put screening methods and demonstrate that changes in pH_{L} are reliably quantified. This quantification is necessary to predict the potential effectiveness of acidifying drugs to restore lysosomal enzyme activity.

When a population or subpopulation is found to contain a compound having desired properties, the screening step may be repeated with additional subpopulations containing the desired compound until the population has been reduced to one or a sufficiently small number to permit identification of the compound desired. Standard HTS assays may be miniaturized and automated, e.g., by replacing the standard 96-well plate with a 1536-well plate permitting the easy assay of up to 1500 different compounds. See, e.g., U.S. Pat. Nos. 6,306,659 and 6,207,391. Any suitable HTS system can be used in practicing the invention, and many are commercially available (see, e.g., LEADseeker™, Amersham Pharmacia Biotech, Piscataway, N.J.; PE Biosystem FMAT™ 8100 HTS System Automated, PE Biosystem, Foster City, Calif.; Zymark Corp., Hopkinton, Mass.; Air Technical Industries, Mentor, Ohio; Beckman Instruments, Inc. Fullerton, Calif.; Precision Systems, Inc., Natick, Mass., etc.).

However, the efficient screening for compounds able to restore lysosomal function requires a rapidly-acting alkalizing agent with similar mode of action that can also reduce the rate of outer segment clearance. When tested, A2E increased pH_{L} in ARPE-19 cells by 0.4 units. Holz and colleagues previously reported A2E responses, but the increase in pH_{L} required four weeks of feeding the cells with A2E (14 nM) every 3-4 days, and the A2E was complexed to low-density lipoprotein (LDL; 10 μg/ml) (Holz et al., supra, 1999). However, as determined in the present study, complexing A2E to LDL did not enhance the effect of A2E in the current trials. In fact, as shown in FIG. 1A, the LDL itself had an alkalizing effect. To reduce the lengthy time course, higher concentrations of A2E (100 nM) were tested, but the cells were killed over a period of 1-2 weeks. Therefore, alternative methods were needed to permit timely testing of the effect of pH on lysosomal activity in the RPE cells.

Therefore, in an embodiment of the invention, the testing process was significantly advanced when it was determined that tamoxifen rapidly elevated lysosomal pH, with levels reaching a plateau within 10-15 minutes (establishing the time point used in all subsequent measurements). This rapid (<10-15 minute) alkalinization of the RPE cells established a high pH_{L} on which test compounds could be tested for their ability to modulate the pH, as compared with the 4-week, prior art time course of A2E-mediated alkalinization which had been used to achieve similar results. The rise in pH by the present method for increasing pH_{L} was concentration dependent, with EC₅₀=22 μM (FIG. 1B). The “rapid-acting” increase (meaning alkalinization) in pH_{L} produced by 15 μM tamoxifen (produced in <10-15 minutes) was equivalent to that which resulted from the long time course of A2E-mediated alkalinization (14 nM).

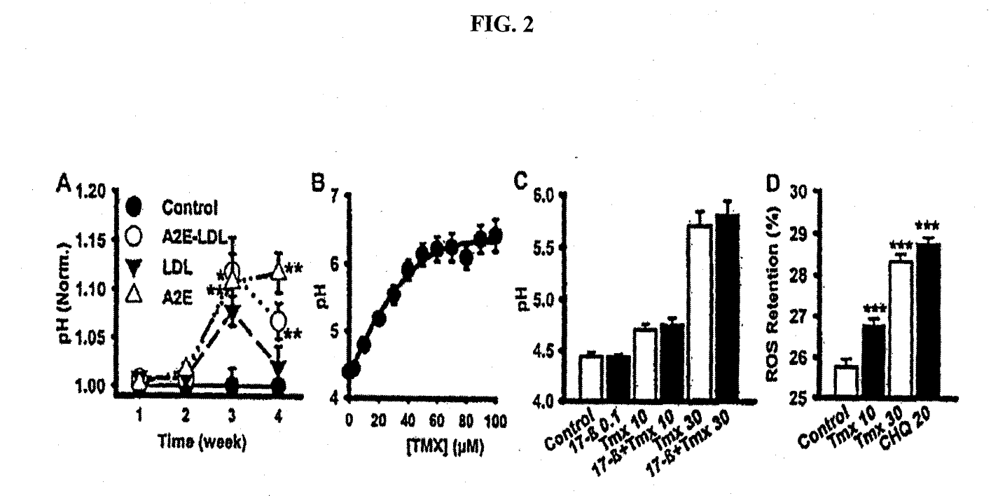

The response to tamoxifen was reversed by the channel blocker 5-nitro-2-(3-phenylpropylamino)-benzoate (“NPPB”), but was neither mimicked, nor inhibited, by 17-13 estradiol (FIG. 1C), indicating that the effect of tamoxifen did not involve estrogen receptors or blockage of channels (Klinge et al., Oncol. Res. 4:137-144 (1992); Zhang et al., J. Clin. Invest. 94:1690-1697 (1994); Valverde et al., Pflug. Archiv. Eur. J. Physiol. 425:552-554 (1993). Tamoxifen slowed the degradation of outer segments at rates approaching chloroquine (FIG. 2D). The reduction in the clearance of outer segments was dose-dependent and proportional to the effect of tamoxifen on pH_{L}, supporting the theory that the two are linked. As a result, although A2E and tamoxifen both elevated the pH_{L} of RPE cells, the discovery of the significantly more rapid action resulting from the use of tamoxifen made this manipulation suitable for rapid screening assays.

High through-put screening methods involve providing a library containing a large number of potential therapeutic compounds (“candidate compounds”) that may be modulators of lysosomal acidity. Libraries of candidate compounds (“combinatorial libraries”) can be screened using one or more assays of the invention, as described herein, to identify those library compounds that display the desired characteristic activity, e.g., modulation of lysosomal activity. A higher or lower level of pH_{L} in the presence of the test compound, as compared with pH_{L} in the absence of the test compound, is an indication that the test compound affects pH_{L}, and therefore, that it also modulates lysosomal activity.

The results are consistent with previous reports, further confirming that tamoxifen alkalizes lysosomes through a detergent-like action (Chen et al., J. Biol. Chem. 274:18364-18373 (1999); Altan et al., Proc. Nat. Acad. Sci. USA 96:4432-4437 (1999)). While the incidence of retinopathies with moderate doses of tamoxifen treatment are low, the problems that occur at higher doses are consistent with increased pH_{L} in the RPE (Lazzaroni et al., Graefes. Arch. Clin. Exp. Ophthalmol. 236:669-673 (1998); Noureddin et al., Eye. 13:729-733 (1999)). The decrease in outer segment clearance in the presence of tamoxifen and/or chloroquine supports the dependence of degradative capacity on pH_{L}, although a direct effect of tamoxifen on lysosomal enzymes may also contribute to the overall effect (Toimela et al. Pharmacol. Toxicol. 83:246-251 (1998); Toimela et al., Ophthal Res. 1:150-153 (1995)). Moreover, these experiments demonstrated the feasibility of measuring both pH_{L} and outer segment clearance using the high through-put screening protocol of the present invention, wherein quantifying the effectiveness of drugs to restore pH_{L} and clearance rates is needed.

Receptor-Mediated Restoration of pH_{L}: Because identifying a drug capable of acidifying distressed lysosomes in RPE cells holds therapeutic potential for treating AMD, the effect of purinergic signaling to RPE physiology was determined. The present findings demonstrated that purines can be used to restore pH_{L}. Low doses of adenosine and the stable adenosine receptor agonist 5′-(N-ethylcarboxamido) adenosine (NECA) were independently administered to the RPE cells and found to reduce the pH_{L} in cells treated with tamoxifen when each compound was given 15 minutes before measurements were made. A delivery for “prolonged period” or “sustained period” of time for the purposes of this invention means >1 hour; >12 hours, >18 hours, >24 hours, 1-3 days, 1-7 days, >1 week, 12 days, >1-2 weeks, to 1 month or more. However, the response to adenosine was more variable (FIG. 2A) than the effect of NECA. While not wishing to be bound by any theory, this is likely because at low concentrations, NECA activates both Aₗ and A_{2A} adenosine receptors (Fredholm et al., Pharmacol. Rev. 46:143-156 (1994)).

Agonists for the A₁ adenosine receptor N⁶-cyclopentyl-adenosine (CPA) and (2S)-N⁶-[2-endo-norbornyl] adenosine (ENBA) had no effect (see, FIG. 2B), the A_{2A} receptor agonist, CGS21680, acidified the lysosomes at levels found previously to be specific ((Mitchell et al., supra, (1999)); FIG. 2C). Over half of the increase triggered by 10 μM tamoxifen was reversed by CGS21680, demonstrating that the compound would largely restore lysosomal acidity to cells challenged with A2E. Message for the A_{2A} adenosine receptor was identified in both ARPE-19 cells and fresh human RPE cells with RT-PCR (FIG. 2D). NECA and adenosine also decreased pH_{L} in primary cultures of bovine RPE cells treated with tamoxifen (FIG. 2E).

Consequently, it was determined that stimulation of adenosine receptors did, in fact, restore pH_{L} in cells treated with tamoxifen, and likely involves the A_{2A} receptor. The acidification of pH_{L} in bovine cells treated with tamoxifen further showed that the responses to tamoxifen are neither species specific, nor restricted to a particular cell line.

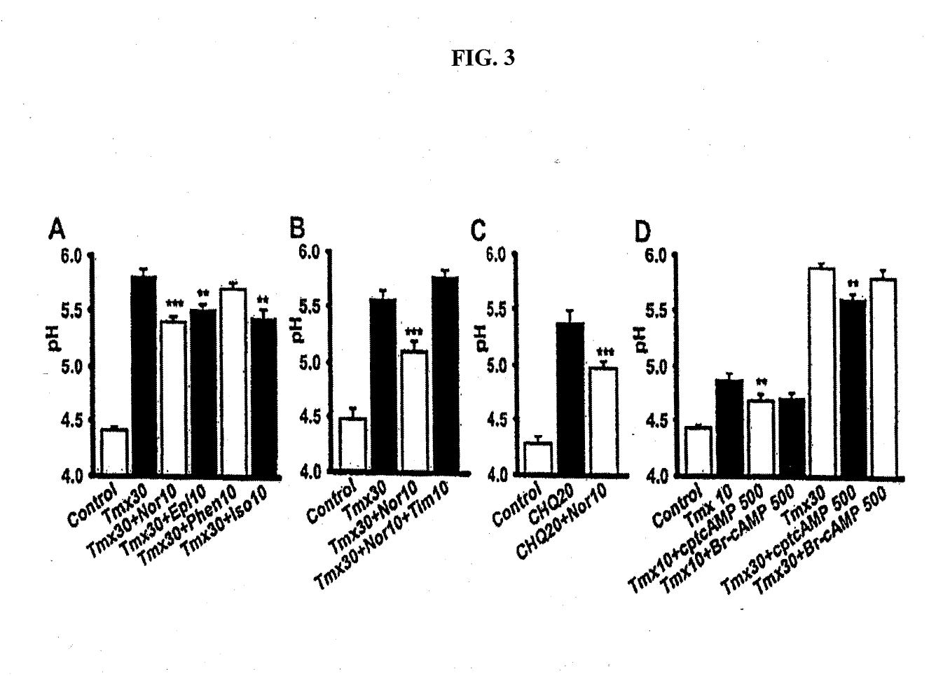

Given that β-adrenergic receptor and cAMP lower lysosomal pH: The acidification of pH_{L} by adenosine and ATP prompted screening for additional compounds. Drugs currently used for ophthalmic treatment and those known to stimulate classic pharmacologic pathways were examined. However, compounds currently in ophthalmic use, including dorzolamide, timolol or latanaprost, did not lower pH_{L} in ARPE-19 cells treated with 30 μM tamoxifen. Conversely, norepinephrine, epinephrine and isoproterenol did significantly decrease pH_{L} (FIG. 3A). Potential second-messenger involvement was also probed to suggest general mechanisms of acidification. As a result, it was determined that phenylephrine had no significant effect on pH_{L}, but the reduction triggered by norepinephrine was blocked by timolol, implying involvement of the β-adrenergic receptor (FIG. 3B).

Since the A_{2A} adenosine and β-adrenergic receptors can act by stimulating Gs, the effect of cAMP was examined directly with cell-permeable forms of cAMP (FIG. 3D). 8-(4-chlorophenylthio) adenosine-3′, 5′-cyclic monophosphate (cpt-cAMP) significantly decreased pHL in cells exposed to 30 and 10 μM tamoxifen, respectively. 8-bromo-adenosine 3′,5′-cyclic monophosphate (8-Br-cAMP) also seemed to acidify lysosomes treated with 10 μM tamoxifen, but the effect was not significant (p=0.054).

Thus, the ability of cpt-cAMP to lower pHL, in conjunction with actions of isoproterenol and CGS21680, indicated that cAMP is a primary regulator of pHL in RPE cells. The magnitude of the acidification is predicted to restore pHL from 4.9 to 4.6 in cells treated with A2E. This corresponds to a predicted increase in activity of cathepsin D from 25% to 60% of maximum rate (Barrett, supra, 1977).

The compounds identified by the methods embodied herein, must be pharmacologically acceptable, but they may be protein or non-proteinaceous, organic or non-organic, and they may be administered exogenously or expression may be up-regulated in the patient. In the alternative, proteinaceous compounds may be produced in vitro, including by recombinant methods, and then administered to the patient,

For proteinaceous compounds, the desired expression products may be generated from transgenic constructs, comprising an isolated nucleic acid or amino acid sequence of the composition, or an active fragment thereof, that lowers pH_{L} in RPE cells and/or restores the degradative capability of the perturbed lysosomal enzymes. The terms “nucleotide molecule,” “nucleotide sequence,” “nucleic acid molecule” and “polynucleotide” are used interchangeably and refer to a polymeric form of nucleotides of any length, either DNA, RNA or analogs thereof. Non-limiting examples of polynucleotides include a gene, a gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes and primers (linear or circular). Amino acid sequences refer to “proteins” or “peptides” as used herein is intended to include protein fragments, or peptides. Thus, the term “protein” is used synonymously with the phrase “peptide” or “polypeptide,” and includes “active fragments thereof,” particularly with reference to proteins that are “proteins of interest.” Protein fragments may or may not assume a secondary or tertiary structure. Protein fragments may be of any length, from 2, 3, 5 or 10 peptides in length up to 50, 100, or 200 peptides in length or more, up to the full length of the corresponding protein.

“Library,” refers to a collection of different compounds, including small organic compounds or biopolymers, including proteins and peptides. The compounds may be encoded and produced by nucleic acids as intermediates, with the collection of nucleic acids also being referred to as a library. When a nucleic acid library is used, it may be a random or partially random library, as in a combinatorial library, or it may be a library obtained from a particular cell or organism, such as a genomic library or a cDNA library. Small organic molecules can be produced by combinatorial chemistry techniques as well. Thus, in general, such libraries comprise are organic compounds, including but not limited oligomers, non-oligomers, or combinations thereof. Non-oligomers include a wide variety of organic molecules, such as heterocyclics, aromatics, alicyclics, aliphatics and combinations thereof, comprising steroids, antibiotics, enzyme inhibitors, ligands, hormones, drugs, alkaloids, opioids, benzodiazepenes, terpenes, prophyrins, toxins, catalysts, as well as combinations thereof. Oligomers include peptides (that is, oligopeptides) and proteins, oligonucleotides (the term oligonucleotide also referred to simply as “nucleotide,” herein) such as DNA and RNA, oligosaccharides, polylipids, polyesters, polyamides, polyurethanes, polyureas, polyethers, poly (phosphorus derivatives), such as phosphates, phosphonates, phosphoramides, phosphonamides, phosphites, phosphinamides, etc., poly (sulfur derivatives), such as sulfones, sulfonates, sulfites, sulfonamides, sulfenamides, etc.

A “substantially pure” or “isolated nucleic acid,” as used herein, refers to a nucleic acid sequence, segment, or fragment which has been separated (purified) from the sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term vector includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like. Suitable vectors also include, but are not limited to, plasmids containing a sense or antisense strand placed under the control of the strong constitutive promoter or under the control of an inducible promoter. Methods for the generation of such constructs are well known in the art once the sequence of the desired gene is known. Suitable vector and gene combinations will be readily apparent to those of skill in the art.

A nucleic acid encoding the therapeutic compound, or an active fragment thereof, can be duplicated using a host-vector system and traditional cloning techniques with appropriate replication vectors. A “coding sequence” or a sequence which “encodes” the selected polypeptide (its “expression product”), is a nucleotide molecule which is transcribed (in the case of DNA) and translated (in the case of mRNA) into a polypeptide, for example, in vivo when placed under the control of appropriate regulatory sequences (or “control elements”). An “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses that incorporate the recombinant polynucleotide. A recombinant polynucleotide may also serve a non-coding function (e.g., promoter, origin of replication, ribosome-binding site).

A “host-vector system” refers to host cells, which have been transfected with appropriate vectors using recombinant DNA techniques. The vectors and methods disclosed herein are suitable for use in host cells over a wide range of eukaryotic organisms. This invention also encompasses cells transformed with the replication and expression vectors, using methods known in the art. Indeed, a gene encoding the modulating nucleic acid, such as the nucleic acid sequence encoding a peptide, or an active fragment thereof, that lowers pH_{L} in RPE cells and/or restores the degradative capability of the perturbed lysosomal enzymes, can be duplicated in many replication vectors, and isolated using methods described, e.g., in Maniatis et al., Molecular Cloning: A Laboratory Manual, Cold Spring Harbor Laboratory, New York (1982) and Sambrook et al., Molecular Cloning: A Laboratory Manual, Cold Spring Harbor Laboratory, New York (1989), and the various references cited therein.

The selected gene, made and isolated using the above methods, can be directly inserted into an expression vector, such as pcDNA3 (Invitrogen) and inserted into a suitable animal or mammalian cell. In the practice of one embodiment of this invention, the gene or gene fragment, such as the purified nucleic acid molecule encoding the peptide, or an active fragment thereof, that lowers pH_{L} in RPE cells and/or restores the degradative capability of the perturbed lysosomal enzymes, is introduced into the cell and expressed. A variety of different gene transfer approaches are available to deliver the gene or gene fragment encoding the modulating nucleic acid into a target cell, cells or tissues.

As used herein, “recombinant” is intended to mean that a particular DNA sequence is the product of various combination of cloning, restriction, and ligation steps resulting in a construct having a synthetic sequence that is indistinguishable from homologous sequences found in natural systems. Recombinant sequences can be assembled from cloned fragments and short oligonucleotides linkers, or from a series of oligonucleotides. As noted above, one means to introduce the nucleic acid into the cell of interest is by the use of a recombinant expression vector. “Recombinant expression vector” is intended to include vectors, capable of expressing DNA sequences contained therein, where such sequences are operatively linked to other sequences capable of effecting their expression. It is implied, although not always explicitly stated, that these expression vectors must be replicable in the host organisms, either as episomes or as an integral part of the chromosomal DNA. Suitable expression vectors include viral vectors, e.g., adenoviruses, adeno-associated viruses, retroviruses, cosmids and others, typically in an attenuated or non-replicative form. Adenoviral vectors are a particularly effective means for introducing genes into tissues in vivo because of their high level of expression and efficient transformation of cells, both in vitro and in vivo.

Accordingly, when reference is made herein to “administering” the compound that lowers pH_{L} in RPE cells and/or restores the degradative capability of the perturbed lysosomal enzymes, or a functionally equivalent peptide fragment thereof, to a patient, it is intended that such methods include not only delivery of an exogenous composition to the patient, but also methods for reducing lysosomal pH (i.e., increasing acidity) within the RPE cells of the patient, or reducing levels of lipofuscin or slowing the rate of lipofuscin accumulation. As noted, the compound may be protein in nature or non-protein. However, when the compound is an expressed protein, expression levels of the gene or nucleotide sequence inside a target cell are capable of providing gene expression for a duration and in an amount such that the nucleotide product therein is capable of providing a therapeutically effective amount of gene product or in such an amount as to provide a functional biological effect on the target cell. By “gene delivery” is meant transportation of a composition or formulation into contact with a target cell so that the composition or formulation is capable of being taken up by means of a cytotic process into the interior or cytoplasmic side of the outermost cell membrane of the target cell, where it will subsequently be transported into the nucleus of the cell in such functional condition that it is capable of achieving gene expression.

By “gene expression” is meant the process, after delivery into a target cell, by which a nucleotide sequence undergoes successful transcription and translation such that detectable levels of the delivered nucleotide sequence are expressed in an amount and over a time period that a functional biological effect is achieved. “Gene therapy” encompasses the terms gene delivery and gene expression. Moreover, treatment by any gene therapy approach may be combined with other, more traditional therapies.

The compounds used for therapeutic purposes are referred to a “substantially pure,” meaning a compound, e.g., a protein or polypeptide which has been separated from components which naturally accompany it. Typically, a compound is substantially pure when at least 10%, or at least 20%, or at least 50%, or at least 60%, or at least 75%, or at least 90%, or at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

By “patient” or “subject” is meant any vertebrate or animal, preferably a mammal, most preferably a human, that is affected by or susceptible to retinal diseases or disorders resulting in macular degeneration and loss of vision. Thus, included within the present invention are animal, bird, reptile or veterinary patients or subjects, the intended meaning of which is self-evident. The methods of the present invention are useful in such a patient for the treatment or prevention of the following, without limitation: macular degeneration, age related macular degeneration, lysosomal alkylinization of the RPE cells of the eye, damaging accumulation of lipofuscin, and other diseases of the retina of the eye.

In another embodiment, the invention may further include the step of administering a test compound to the cell prior to the detecting step, wherein the absence of binding of the detectable group to the internal structure indicates that the test compound inhibits the binding of the members of the specific binding pair. Any test compound can be used, including peptides, oligonucleotides, expressed proteins, small organic molecules, known drugs and derivatives thereof, natural or non-natural compounds, non-organic compounds, etc. Administration of the test compound may be by any suitable means, including direct administration, such as by electroporation or lipofection if the compound is not otherwise membrane permeable, or (where the test compound is a protein), by introducing a heterologous nucleic acid that encodes and expresses the test compound into the cell. Such methods are useful for screening libraries of compounds for new compounds that disrupt the binding of a known binding pair.

In yet another embodiment, the present invention provides an assay for determining agents, which stimulate dopamine receptors to modify pH of the retinal pigment epithelial lysosomes (pH_{L}), or that bind to, neutralize or acidify lysosomes of the RPE, or other factors in a sequence of events leading to the onset of lysosomal alkylinization of the RPE cells of the eye, damaging accumulations of lipofuscin, and eventually macular degeneration, thereby reducing, modulating or preventing such pathologies. Such an assay comprises administering an agent under test to the cells or model animals, such as those described herein, at low cell density, and monitoring the onset of lysosomal alkylinization of the RPE cells of the eye or whether the agent effects a reversal of the problem. For example, Lysosensor Yellow/Blue is an effective method of quantifying pH_{L} in RPE cells. A further assay according to the invention comprises administering the agent under test to determine and measure the reduction in outer segment degradation triggered by the agent. Agents may thus be selected which effectively reduce, inhibit, neutralize or prevent lysosomal alkylinization of the RPE cells, retinal dysfunction, or the like. The agents thus selected, and the assays used to identify them, are also intended to be a part of the present invention.

In still another embodiment, sensitivity of pH_{L} levels in vivo are used as a biomarker for measuring macular disease severity or treatment effectiveness.

In accordance with the present invention, the compound (including organic or non-organic compositions, a peptide, receptor, or an active fragment thereof), that lowers pH_{L} in RPE cells and/or restores the degradative capability of the perturbed lysosomal enzymes, or fragment thereof, or that binds to, neutralize or inhibit lysosomal alkylinization of the RPE cells, when used in therapy, for example, in the treatment of an aging patient or one with early onset symptoms of macular degeneration, lysosomal alkylinization of the RPE cells, damaging accumulations of lipofuscin, retinal dysfunction, or the like, can be administered to such a patient either alone or as part of a pharmaceutically acceptable composition. Optionally with a preservative, diluent, and the like are also added. The compound may further be administered in the form of a composition in combination with a pharmaceutically acceptable carrier or excipient, and which may further comprise pharmaceutically acceptable salts. Examples of such carriers include both liquid and solid carriers, such as water or saline, various buffer solutions, cyclodextrins and other protective carriers or complexes, glycerol and prodrug formulations. Combinations may also include other pharmaceutical agents.

The term “pharmaceutically acceptable” refers to physiologically and pharmaceutically acceptable compounds of the invention: i.e., those that retain the desired biological activity and do not impart undesired toxicological effects on the patient or the patient's eye or RPE cells.

Various methods of “administration” of the therapeutic or preventative agent (compound or composition) can be used, following known formulations and procedures. Although targeted administration is described herein and is generally preferred, it can be administered intravenously, intramuscularly, subcutaneously, topically, intraorbitally, optionally in a dispersible or controlled release excipient. One or several doses may be administered as appropriate to achieve systemic or parental administration under suitable circumstances. Compounds or compositions suitable for parenteral injection may comprise physiologically acceptable sterile aqueous or nonaqueous solutions, dispersions, suspensions, or emulsions, and sterile powders for reconstitution into sterile injectable solutions or dispersions. Examples of suitable aqueous and nonaqueous carriers, diluents, solvents, or vehicles include water, saline, buffered saline, dextrose, ethanol, glycerol, polyols, and the like, and suitable mixtures thereof. Proper fluidity can be maintained, for example, by the use of a coating, such as lecithin, by the maintenance of the required particle size in the case of dispersions and by the use of surfactants. These compositions may also contain adjuvants, such as preserving, wetting, emulsifying, and dispensing agents. Sterility can be ensured by the addition of various antibacterial and antifungal agents. It may also be desirable to include isotonic agents, for example sugars, sodium chloride and the like. Prolonged absorption of the injectable pharmaceutical form can be brought about by the use of agents delaying absorption, for example, aluminum monostearate and gelatin.

Persons of ordinary skill can easily determine optimum dosages, dosing methodologies and repetition rates. Repetition rates for dosing can be readily estimated based upon measured residence times and concentrations of the drug in bodily fluids or tissues. Amounts and regimens for the administration of compounds used to lower pH_{L} in RPE cells and/or restores the degradative capability of the perturbed lysosomal enzymes can be determined readily by those with ordinary skill in the clinical art of treating retinal disease, including macular degeneration. Generally, the dosage of such compounds or treatment using such compounds will vary depending upon considerations, such as: age; health; conditions being treated; kind of concurrent treatment, if any, frequency of treatment and the nature of the effect desired; extent of tissue damage; gender; duration of the symptoms; and, counter-indications, if any, and other variables to be adjusted by the individual physician. Dosage can be administered in one or more applications to obtain the desired results (see, e.g., dosages proposed for human therapy in known references).

When the therapeutic compound is a peptide, or an active fragment thereof, that stimulates a dopamine receptor to modify pH_{L}, lowers pH_{L} in RPE cells and/or restores the degradative capability of the perturbed lysosomal enzymes, instead of direct administration to the target cells, such peptides can also be produced in the target cells by expression from an encoding gene introduced into the cells, e.g., in a viral vector. The vector could be targeted to the specific cells to be treated, or it could contain regulatory elements, such as receptors, which are switched on more or less selectively by the target cells. Increased expression is referred to as “up-regulation” as discussed herein.

By “therapeutically effective” as used herein, is meant that amount of composition that is of sufficient quality and quantity to neutralize, ameliorate, modulate, or reduce the cause of or effect of lysosomal alkylinization of the RPE cells, retinal dysfunction, macular degeneration or the like.

By “ameliorate,” “modulate,” or “decrease” is meant a lessening or lowering or prophylactic prevention of the detrimental effect of the disorder in the patient receiving the therapy, thereby resulting in “protecting” the patient. A “sufficient amount” or “effective amount” or “therapeutically effective amount” of an administered composition is that volume or concentration which causes or produces a measurable change from the pre-administration state in the cell or patient, this is also referred to herein as “restoring” or “restoration of” the lysosomal acidity.

While the subject of the invention is preferably a human patient, it is envisioned that any animal with lysosomal alkylinization of the RPE cells, damaging accumulations of lipofuscin, retinal dysfunction, macular degeneration or the like, can be treated by a method of the present invention. As used herein, the terms “treating” and “treatment” are intended to include the terms “preventing” and “prevention.” One embodiment of the present invention includes the administration of a compound (including an organic or inorganic composition, peptide, or an active fragment thereof, receptor, etc) that stimulates the D5 receptor to to modify pH_{L}, lowers pH_{L} in RPE cells and/or restores the degradative capability of the perturbed lysosomal enzymes, in an amount sufficient to treat or prevent lysosomal alkylinization of the RPE cells, lipofuscin accumulation, retinal dysfunction, macular degeneration, or the like.

The terms “inhibition” or “blocking” refer to a statistically significant decrease in lysosomal alkylinization of the RPE cells or lipofuscin accumulation, associated with retinal dysfunction, macular degeneration, or the like, as compared with a selected standard of activity or for cells or tissues grown without the addition of the selected compound (including a peptide, or an active fragment thereof) that lowers pH_{L} in RPE cells and/or restores the degradative capability of the perturbed lysosomal enzymes. “Preventing” refers to effectively 100% levels of prophylactic inhibition. Preferably, the increased levels of the compound (meaning a higher concentration than was present before additional quantities of the compound was administered or before its expression was up-regulated in the patient) decreases lysosomal alkylinization of the RPE cells or lipofuscin accumulation, associated with retinal dysfunction, macular degeneration, or the like, or risk thereof, by at least 5%, or by at least 10%, or by at least 20%, or by at least 50%, or even by 80% or greater, and also preferably, in a dose-dependent manner.

The invention is further defined by reference to the following specific, but nonlimiting, examples that describe stimulation of the D5 receptor to modify pH_{L}, reverse or alter lysosomal alkylinization of the RPE cells or change lipofuscin accumulation, associated with retinal dysfunction, macular degeneration, or the like. Reference is made to standard textbooks of molecular biology that contain definitions and methods and means for carrying out basic techniques, encompassed by the present invention. It will be apparent to one skilled in the art that many modifications, both to materials and methods, may be practiced without departing from the purpose or narrowing the scope of this invention.

### EXAMPLES

Materials and Methods: The following Materials and Methods apply to all of the following Examples of the present invention.

ARPE-19 cells: ARPE-19 cells (ATCC) were grown to confluence in 25 cm² Primary Culture flasks (Becton Dickinson) in a 1:1 mixture of Dulbecco's modified Eagle medium (DMEM) and Ham's F12 medium with 3 mM L-glutamine, 100 U/mL streptomycin or penicillin, 100 μg/ml streptomycin, and 2.5 mg/ml Fungizone and/or 50 μg/ml gentamicin and 10% fetal bovine serum (all Invitrogen Corp). Cells were incubated at 37° C. in 5% CO₂, and subcultured weekly with 0.05% trypsin and 0.02% EDTA. In many experiments, cells were grown for 2 weeks, with the above growth medium replaced with one containing only 1% serum for the second week to encourage differentiation.

Isolation of bovine and mouse RPE cells: The bovine RPE-choroid and sclera were removed, incubated in 2.5% trypsin at 37° C. in 5% CO₂ for 30 min, after which RPE sheets are dissected, washed and plated in 96-well plates with 10% serum medium. Mouse eyes were incubated in DMEM for 3 hrs at room temperature (RT), then in 0.1% trypsin and 0.4 mg/ml collagenase IV with 1 mM EDTA for 45 min at RT. RPE sheets were dissected out, washed, and incubated with 0.25% trypsin/ 0.02% EDTA in order to obtain a suspension of single cells, then grown as above.

HTS measurement of pH_{L}: ARPE-19 cells were grown in 96-well plates, rinsed 3× with isotonic solution (IS; prepared from NaCl 105 mM, KCl 5 mM, HEPES Acid 6 mM, Na HEPES 4 mM, NaHCO₃ 5 mM, mannitol 60 mM, glucose 5 mM, MgCl₂ 0.5 mM, CaCl₂ 1.3 mM) and incubated with 5 μM LysoSensor Yellow/Blue (Invitrogen Corp.) diluted with IS. Extensive trials determined that the optimal response is obtained with 5 minute dye loading and 15 minute post-incubation (Liu et al. 2008). Fluorescence was measured with a Fluroskan 96-well Plate Reader (Thermo Electron Corp.). pH_{L} was determined from the ratio of light excited at 340 nm vs 380 nm (>520 nM em). pH_{L} was calibrated by exposing cells to 10 μM H⁺/Na⁺ ionophore monensin and 20 μM H⁺/K⁺ ionophore nigericin in 20 MES, 110 KCl and 20 NaCl at pH 4.0-6.0 for 15 min. All reagents were from Sigma Chemical Corp. unless otherwise indicated.

Measurement of pH_{L} from isolated mouse cells: Based on protocols that are used extensively to measure Ca2⁺ from retinal ganglion cells (Zhang et al. Invest. Ophthalmol. Vis. Sci. 46:2183-2191 (2005)), cells were fixed on coverslips and mounted on Nikon Eclipse inverted microscope, visualized with a x40 oil-immersion fluorescence objective, and perfused with control solution. The field was alternatively excited at 340 nm and 380 nm, and fluorescence >515 emitted from the region of interest surrounding individual cells is measured with a CCD camera and Imagemaster software (Photon Technologies International, Inc). After baseline levels were recorded for 3-5 minutes in the absence of dye, solution was replaced with 5 μM Lysosensor Yellow/Blue dye for 5 minutes before washing for an additional 15 minutes. The ratios in the control solutions were recorded, and then acidifying drugs were added. Ratios were converted to pH with monensin/nigericin as above.

siRNA silencing of D1 or D5 receptors: D1DR and D5DR expression was silenced using manufacturer's protocols. ARPE-19 cells were transfected with siRNAs specific for DRD1 receptor (s4283) or DRD5 receptor (s4291) purchased from Ambion, and 70-80% confluent ARPE-19 grown in 25 cm2 flasks were transfected with siRNA using Amaxa Cell Line Nucleofector Kit V (VCA 1003, Lonza, N.J., USA). 106 cells were used per condition. Cells transfected with scrambled siRNA (Silencer negative control 1, catalog number 4611; Ambion, Austin, Tex., USA) served as a negative control. As an additional control, cells were mock-transfected using transfection reagent alone. The D1, D5, or scrambled siRNA was used at a final concentration of 300 nM. Lysosomal pH was determined 72 h after transfection.

Western blots: The term “Western blot,” refers to the immunological analysis of protein(s), polypeptides or peptides that have been immobilized onto a membrane support. ARPE-19 cells were lysed in RIPA buffer (150 mM NaCl, 1.0% Triton X-100, 0.5% Na-Deoxycholate, 0.1% SDS, 50 mM Tris, pH 8.0, and protease inhibitor cocktail) and centrifuged at 13000 g for 10 mM at 4° C. Protein concentrations were determined using the BCA kit (Pierce, Rockford, Ill., USA). Protein lysates were loaded in each lane in sample buffer (2% SDS, 10% glycerol, 0.001% bromophenol blue, and 0.05 M Tris-HCl, pH 6.8), separated on SDS-PAGE (Biorad, Hercules, Calif., USA) and transferred to polyvinylidene fluoride membrane (Millipore Corporation, Bedford, Mass., USA). For identification of the dopamine receptors, 35 μg protein was run on a 10% gel, blots were blocked with 5% non-fat milk in PBS and incubated overnight with rabbit anti-D5DR (1:2000) or mouse anti-D1DR (1:1000; both Santa Cruz Biotechnology, Calif., USA). Mouse anti-b-actin was used as a control for normalizing (1:1000; Sigma, St. Louis, Mo., USA). Visualization of the primary antibody was performed by incubating membranes with the corresponding peroxidase-conjugated secondary antibody (1:3000; GE Healthcare, Waukesha, Wis., USA) for 1 h at 25° C. Finally, the blots were developed by enhanced chemiluminescence (ECL; Amersham Pharmacia Biotech, now GE Healthcare, Piscataway, N.J., USA) and captured on an ImageQuant LAS 400 image reader (GE Healthcare). Bands were quantified using the Alphaimager HP gel documentation system (ProteinSimple, Santa Clara, Calif., USA).

POS membrane preparation: Fresh bovine retinas were isolated in the light under sterile conditions as previously described (Boesze-Battaglia and Yeagle 1992). Thawed retinas were agitated in 30% (w/w) buffered sucrose solution (containing 5 mM HEPES pH 7.4, 65 mM NaCl, 2 mM MgCl₂) followed by centrifugation in a Sorvall SS-34 rotor (7 min, 700 rpm, 4° C.). The supernatant was diluted in two volumes of 10 mM HEPES pH 7.4 and further centrifuged (Sorvall SS-34 rotor, 20 min, 3600 g, 4° C.). The resulting pellet was then homogenized and layered on top of a discontinuous sucrose density gradient. Density gradient solutions of 36, 32, and 26% sucrose (w/w) were employed, and POS membranes were harvested from the 26%/32% sucrose solution interface (Papermaster and Dreyer 1974). POS prepared this way was washed in three volumes of 0.02 M Tris buffer, pH 7.4 (Sorvall SS-34 rotor, 10 min, 20 000 g, 4° C.). The pellet was resuspended in 2.5% (w/w) buffered sucrose solution and POS stored at −80° C.

Outer segment degradation: Bovine retinas were homogenized in 20% sucrose with 130 mM NaCl, 20 mM Tris-HCl, 10 mM glucose, 5 mM taurine and 2 mM MgCl₂ (pH 7.20). The homogenate was placed in ultracentrifuge tubes with 20%, 27%, 33%, 41%, 50% and 60% sucrose, respectively, and centrifuged for 70 minutes at 28,000 rpm on a SW28 rotor (4° C.). The supernatant was filtered, diluted in 0.02M Tris-HCl buffer (pH 7.2) and centrifuged at 13,000×g for 10 minutes (4° C.). The pellet was resuspended in 10 PBS, 0.1 mM NaCl and 2.5% sucrose. Outer segments were loaded with 5 μM calcein-AM in PBS for 10 minutes, and spun 2× at 14,000 rpm to wash. Outer segments were then diluted 1:100 in growth medium and added to ARPE-19 cells in 96-well plates. After 2 hours, cells were washed vigorously 3×, and incubated with growth medium for 3 hours, after which 30 μM tamoxifen was added with acidifying drugs. After 24 hours, wells were washed 3×, and the fluorescence was read with a plate scanner at 485 nm to quantify the signal.

Visualization of cellular autofluorescence: ARPE-19 cells were plated to confluence on 12 mm cover slips. The cells were then incubated without or with POS (106/mL) for 7 days. Culture medium and POS were renewed every alternate day during this time. After the final incubation, cells were washed to remove the non-internalized POS, and after waiting for a 2 h “chase” period for remaining material to be internalized, cells were fixed with paraformaldehyde and stained with DAPI for 1 min to visualize the nuclei. For localization of POS-associated autofluorescence, cells exposed to the outer segments for 7 days were incubated in 5 μM LysoTracker Red DND-99 (Invitrogen Corp) in cell culture medium for 15 min. Cells were washed again before imaging with a Nikon Al inverted confocal microscope. Images were acquired and processed with NIS-Elements software (Nikon Instruments Inc., Melville, N.Y., USA).

Flow cytometry: ARPE-19 cells were grown to confluence in 6-well plates and incubated with POS (106/mL) for 2 h (pulse); the cells were washed thoroughly to remove non-internalized POS followed by a 2-h chase. Subsequently, the cells were incubated with and without 10 μM SKF 81297. Culture medium and POS were renewed every alternate day for 7 days. For flow cytometric quantification of lipofuscin-like autofluorescence, cells were repeatedly washed, detached with trypsin, and analyzed on one of two flow cytometers (FACS Calibur, BD Biosciences, Heidelberg, Germany or LRSII, BD Biosciences, Franklin Lakes, N.J., USA) using the FITC channel (excitation laser wavelength, 488 nm; detection filter wavelength, 530/30 nm). Cell debris and cell clusters were identified and excluded from the run analysis using FTC and SSC. Over 10 000 gated events were recorded.

Assessment of degradative enzyme activity using BODIPY: FL-pepstatin A probe Cathepsin D activity was measured with the fluorescent probe BODIPY FL-pepstatin A (Invitrogen). The probe itself is synthesized by covalently conjugating the BODIPY (Boron dipyrromethene difluoride) fluorophore to pepstatin A, a potent and selective inhibitor of cathepsin D. As the probe binds to the active site of cathepsin D, fluorescence intensity provides a measure of the activity of cathepsin D. To quantify cathepsin D activity, cells were grown to confluence on black-walled, clear-bottomed 96-well plates until confluent, and then incubated for 48 h in either control culture medium, 10 μM CHQ in medium, or 10 μM CHQ+10 μM SKF 81297. Cells were then incubated in 1 μM BODIPY probe at 37° C. in the dark. After washing, fluorescence was quantified using a Fluoroskan plate reader at 485 nm/527 nm (ex/em). Background fluorescence was subtracted from the plates.

Isolation and measurement of lysosomal pH from fresh ABCA4^{-/-} mouse RPE cells: ABCA4^{-/-} mice were reared at 5-15 lux and killed with a CO₂ overdose. Mouse eyes were isolated and processed as described (Liu et al., supra, 2008). In brief, after enucleation, intact eyes were incubated in 2% dispase and 0.4 mg/mL collagenase IV for 45 min, rinsed and incubated in growth medium for 20 min (containing DMEM with 1 MEM+non-essential amino acids, 3 mM 1-glutamine, 100 U/mL penicillin, 100 1g/mL streptomycin, and 2.5 mg/mL Fungizone and/or 50 lg/ mL gentamicin, plus 10% fetal bovine serum; all Invitrogen Corp). In some experiments, the anterior segments and retinas were removed and the eyecup was rinsed with Versene (Dow Chemical Corp., Midland, Mich., USA) and incubated in 0.25% trypsin for 45 min. Sheets of RPE cells were separated from the choroid and triturated into single cells. Cells from two to six eyes were pooled, loaded with 2-5 μM LysoSensor Yellow/Blue for 5 min at RT, rinsed and distributed into wells of 384 well UV Star plates (Greiner Bio-One, Monroe, N.C., USA) and measured as described above. Although eyes from ABCA4^{-/-} mice were slightly autofluorescent, the signal from the dye was 100-fold greater, validating the measurements (Liu et al., supra, 2008). Dopamine agonists were added to the bath 20 min before measurements were taken. Lysosomal pH was measured within 3-h post-mortem. Because of the reduced number of cells, measurements from fresh RPE cells were not calibrated and are expressed as ratio of fluorescence excited at 340 versus 380 nm and emitted >527 nm.

Isolation of lysosomes: ARPE-19 cells were detached with 0.25% trypsin, centrifuged at 1000 rpm for 5 minutes, and resuspended in 0.25M sucrose with 5 mM ATP in 10 mM Tris buffer (pH 7.4 with HCl). After homogenization, samples were spun at 1000×g (10 min). The supernatant was centrifuged (20,000×g, 10 min) and the pellet was resuspended in a 0.25 M sucrose buffer with 8 mM CaCl₂ in Tris-HCl buffer (pH 7.4) to lyse mitochondria (15 min, 35° C.). After a subsequent centrifugation (5000×g, 15 min), the supernatant was placed on top of a discontinuous sucrose gradient (45%, 34.5% and 14.3%, Tris-HCl buffer). The lysosomal fraction was collected in the 34.5%-14.3% interface after an ultracentrifugation at 77,000×g for 2 hours in a SW71 rotor. After isolation, lysosomes were diluted 1:10 in a 150 mM KCl solution in Tris-HCl (pH 7.4) and pelleted at 25,000×g. The pellet was then resuspended in 5 μM Lysosensor dye. Cells were washed 2× by centrifugation (25,000×g, 15 min), resuspended in test or control solutions including 5 mM MgATP, plated into a 96 well plate (50 μl/well) and the pH was measured as above.

### Example 1

### Effect of Lysosomal Acidification on Clearance of Photoreceptor Outer Segments

To show that lowering pH_{L} increased the clearance of outer segments, an approach was designed based upon the findings that tamoxifen and chloroquine slowed the clearance of outer segments. This also showed whether drugs capable of lowering lysosomal pH, also enhance clearance of outer segments. In addition, this experiment provided a second methodology to assess the effectiveness of the compounds identified above.

The primary lysosomal enzymes in RPE cells function optimally in acidic environments, and compounds that alkalize lysosomes can slow the degradation of outer segments and enhance accumulation of undigested material. Because this accumulation appeared to be a key step in the development and accumulation of lipofuscin, the ability of acidifying drugs to also restore rates of outer segment clearance was central to the potential of a drug.

Isolated bovine outer segments loaded with calcein were supplied to ARPE-19 cells in 96-well plates for 2 hrs, washed 3× and maintained in control medium for an additional 3 hr (see Methods). Acidifying drugs were then added at the most effective concentrations as identified above. Drugs were given to cells both with, and without, tamoxifen to determine whether baseline levels of degradation were also altered. Because lipofuscin is distributed heterogeneously across foveal RPE cells in macular degeneration (Holz et al., Invest. Ophthalmol. Vis. Sci. 42:1051-1056 (2001)), drugs with a minimal impact on healthy cells were preferable. As some compounds may have an independent effect on the rates of phagocytosis (Hall et al., Invest. Ophthalmol. Vis. Sci. 34:2392-2401 (1993)), the effect of the signal in the absence of tamoxifen was subtracted from the effect with tamoxifen to isolate specific actions. Promising compounds were examined for their effects on cells treated with A2E, although the restoration of pH_{L} is unlikely to remove A2E itself. However, other components of the outer segments are also amenable to digestion by lysosomal enzymes at the appropriate pH_{L}, and acidification could minimize the secondary effect of this accumulation.

Phagocytosis of photoreceptor outer segments by the RPE involves binding, ingestion and degradation. Binding is distinguished by labeling outer segments with FITC, and quenching any fluorescence remaining on the membrane with trypan blue. While the increased brightness, pH independence, and the minimal background fluorescence with calcein-AM, make the outer segments labeled with calcein preferable in studies of lysosomes, it was determined that calcein is relatively resistant to quenching. However, the effect of binding was minimized by the 3 hour window between exposure to outer segments and the application of drugs, and the measurements taken 24 hrs later. As A2E does not affect binding itself, these precautions enabled the use of calcein with its multiple advantages.

### Example 2

### Restoration of Lysosomal Acidity in ABCA4^{-/-} Mice

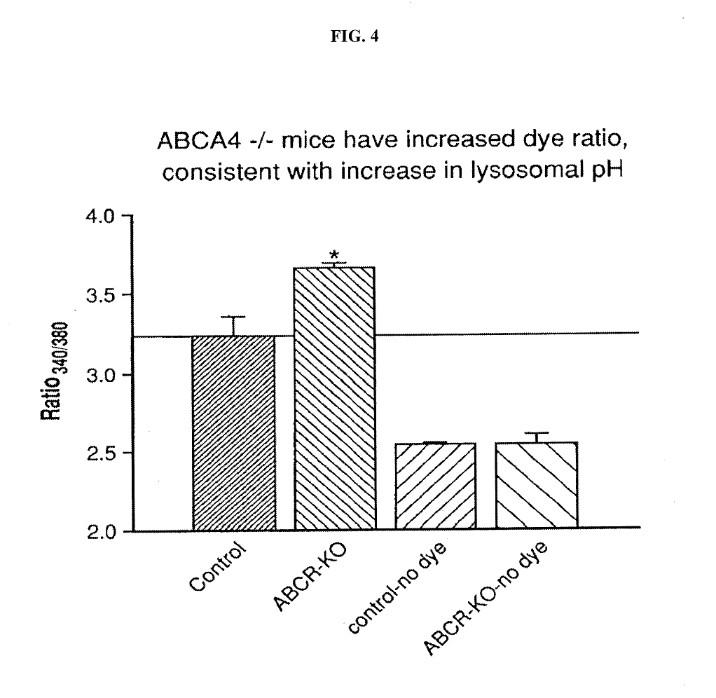

ABCA4^{-/-} mice are missing the gene that is mutated in Stargardt's disease, and share many characteristics with the human form, including increased A2E. As shown in FIG. 4, ABCA4^{-/-} mice had an increased ratio of dye at 340/380 nm, consistent with an increased lysosomal pH, showing that elevated pH occurs in an animal model of Stargardt's disease, representative of a human response, and supporting the concept that lowering pH has direct implications for treating this disease, and by extension, for treating macular degeneration in both the model animal and in humans.

Measurements of lysosomal pH from fresh mouse RPE cells: To verify the effectiveness of the ABCA4^{-/-} model, the LysoSensor Yellow/Blue assay system was tested. LysoSensor Yellow/Blue dye was detected in freshly isolated mouse RPE cells, and first viewed as a brightfield image. The same field was exposed to fluorescence imaging, and excited at 360 nm (em:510 nm). It was, thus, confirmed that the pigment does not interfere with fluorescence. As previously shown, tamoxifen (30 μM) increased the 340/380 nm ratio in isolated mice RPE cells loaded with LysoSensor dye, consistent with the increase in pH found in ARPE-19 cells. This verified the feasibility of measurements from ABCA4^{-/-} mice as an AMD model for experimental purposes. See, FIG. 4.

Restoration of pH_{L} in ABCA4^{-/-} mice: The ABCA4^{-/-} mouse's early onset of A2E accumulation makes the ABCA4^{-/-} mouse an appropriate animal model, demonstrating a progressive accumulation of A2E in its RPE over 18 weeks when housed in 12 hour cyclic light of 25-30 lux (Mata et al., Proc. Nat. Acad. Sci. USA 97:7154-7159 (2000)). As a result, lysosomal pH increases early, and is measured in ABCA4^{-/-} mice at 6, 12 and 18 weeks from RPE cells within 5 hours of sacrifice. As cell division may dilute the lysosomal contents, culturing these cells would diminish the effect on pH. However, the signal/noise from measurements of isolated cells with the plate reader is not acceptable. Instead, this signal is measured using the microscope-based imaging system, previously used successfully to measure Ca.²⁺ from freshly isolated retinal ganglion cells (Zhang et al., supra, 2005).

This system was also used to record pH_{L} from ARPE-19 cells before the high through-put system was developed. Initial readings were made with excitation at 340 and 380 nm in the absence of dye to record any autofluorescence for later subtraction. Next, cells were bathed in 5 μM Lysosensor dye for 5 minutes, followed by 15 minute wash. Baseline pH_{L} was monitored for 3-5 minutes from cells in isotonic solution, after which CFTR activations and other compounds identified above to acidify lysosomes were added at appropriate concentrations. Once a new pH was reached, control solution was returned and the protocol was repeated. The pH_{L} was calibrated at the end of the experiment by perfusing with monensininigericin solutions. Parallel experiments were then performed on ABCA4^{+/+} mice.

Assessment of ABCA4^{-/-} mice: The correct interpretation of the foregoing experiments depends upon assessment of genotype and phenotype. ABCA4^{-/-} mice are bred and housed as described, using protocols established in the inventors' laboratory. Several phenotypic changes have been characterized in ABCA4^{-/-} mice including increases in levels of A2E levels, morphological changes surrounding Bruch's membrane and reduced magnitude of the ERG a-wave maximal response (Weng et al., Cell. 98:13-23 (1999); Mata et al. Invest. Opthalmol. Vis. Sci. 42:1685-1690 (2001)). While it is neither practical nor necessary to repeat all assays, disease progression in the mice is determined as described by performing full field ERGs on age-matched wild type and knockout mice. The time course of the decrease in the a-wave is compared to that published by Travis and colleagues to orient the progression to other phenotypic changes. Thus, these data show that pH_{L} is elevated in ABCA4^{-/-} mice, as compared to control animals, and that pharmacologic manipulation can restore the acidic pH to lysosomes of ABCA4^{-/-} mice.

### Example 3

### Restoring Lysosomal pH

Having previously determined the damaging effect of age-increased pH in RPE cells, specifically in the effect on the ability of the lysosomes to clear spent photoreceptor outer segments and lipofuscin, this experiment focused on how to restore optimal acidic pH to the affected lysosomes in the RPE, and to the identification of drugs or compounds that can achieve that effect and also prevent or restore the damage caused by the increased pH. Further this experiment evaluated the effect of D Nike dopamine receptors and D1-like dopamine receptor agonists, which led to the discovery that the D1-like agonists represent a likely target. This is particularly relevant since the D1-like agonists are also currently being developed to treat Parkinson's disease.

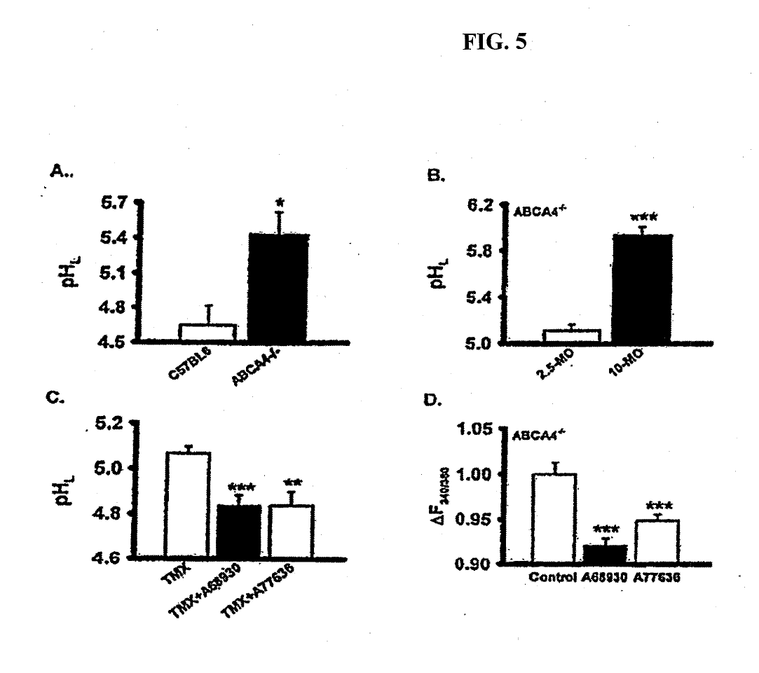

Initially, the magnitude of the damage to lysosomes in RPE cells from the ABCA4^{-/-} mouse model of Stargardt's disease was evaluated. In 6 trials of in RPE cells from ABCA4^{-/-} mice (26 mice aged 216±28 days), as compared to 7 trials in cells from wild type mice (22 mice aged 215±32 days), increased pH_{L} was clearly documented as rising from 4.65 0.17 to 5.43±0.19 units. See, FIG. 5A. This is precisely the range over which degradative lysosomal enzymes lose their function, further linking this defect to the accumulation of partially degraded material found in the RPE of patient's with Stargardt's disease. Lysosomal pH rose with age (FIG. 5B; 4 trials, 2 ABCA4^{-/-} mice each; age shown in months (MO), consistent with both an age-dependent rise in A2E levels and the progression of Stargardt's disease (Mata et al., Invest. Opthamol. Vis. Sci. 42:1685-1690 (2001)).

Recognizing that increased cAMP, and receptors coupled to the Gs protein that leads to elevated cAMP, led to the general conclusion that stimulation of the receptors coupled to the Gs proteins offered a treatment for restoring an acidic pH to the perturbed lysosomes, and thus, for improving degradative function. The most effective receptor is decided by numerous factors, including the availability and side-effects of appropriate agonists to the selected receptor. As such, D1-like dopamine receptors were selected as a particularly well-suited target.

Two specific D1-like agonists A77636 and A68930 (which are also within the subset of D5DR agonists) were then tested and shown to lower lysosomal pH in ARPE-19 cells (FIG. 5C). In 8 tests, dopamine D1-like receptor agonists A68930 (1 μM) and A77636 (1 μM) decreased lysosomal pH of ARPE-19 cells treated by tamoxifen (n=8). In addition, in 8 further tests, the two drugs also restored lysosomal pH in fresh RPE cells from ABCA4^{-/-} mice (FIG. 5D; values are given as the ratio of light excited at 340 to 380 nm, an index of lysosomal pH. *=p<0.05, **=p<0.01, ***=p<0.001 vs control). The mice in these tests were 11 months old, demonstrating that this treatment is effective, even on mice whose lysosomes have been damaged for an extended time. Thus, it is shown that the use of D1-like dopamine agonists is an effective treatment for both Stargardt's disease and macular degeneration. As the RPE cells contain D5 receptors (Versaux-Botteri et al., Neurosci. Letts. 237:9-12 (1997)), these were ultimately a target.

### Example 4

### D1/D5 Receptor Agonists Acidify Compromised Lysosomes.

Next the ability of D1-like receptor agonists (using, e.g., A68930; A77636; and SKF 81297) to lower lysosomal pH in challenged ARPE-19 cells was examined. Baseline pH_{L} levels were typically in the range of 4.5-4.8. Tamoxifen increases lysosomal pH rapidly in various cell types independently of an estrogen receptor, presumably through its actions as both a tertiary amine and by increasing proton permeability (Altan et al., supra, 1999; Chen et al., supra 1999). The pH_{L} of cells exposed to 10 μM tamoxifen for 5 min rose significantly, while the absolute magnitude of the alkalinization varied, the pH_{L} was usually in the range of 5.1-5.3. Chloroquine likewise alkalized lysosomal pH.

The D1-like receptor agonist A68930 led to a substantial acidification of lysosomes in ARPE-19 cells challenged by challenged by 10 μM of the lysotropic agent tamoxifen (TMX) (n=14-40). See FIG. 6A. The effect was rapid, with stable reacidified pH_{L} levels observed within 10 min of drug application. A reduction in lysosomal pH was observed with 1 μM, but did not increase with concentration, perhaps because of the ability of increasing levels to stimulate D2-like receptors (DeNinno et al., supra (1991)). The other exemplary D1-like receptor agonists A77636 (FIG. 6B) and SKF 81297 (FIG. 6C) were also effective when used at 10 μM to rapidly acidifying lysosomes (within 10 min, or less) that had been exposed to 10 μM tamoxifen.

While all three D1-like receptor agonists displayed at least some efficacy in restoring lysosomal pH, additional experiments were performed using SKF 81297 as it displayed a relatively high selectivity for D1-like receptors, as compared with D2-like receptors (Andersen and Jansen, Eur. J. Pharmacol. 188:335-347 (1990)), and it gave the most consistent results in the trials. The ability of SKF 81297 to acidify compromised lysosomes was inhibited by myristoylated protein kinase inhibitor PKI (14-22) amide (100 μM), the cell-permeant inhibitor of protein kinase A (PKA) (FIG. 6D). PKI blocked the effects of SKF 81297 (10 μM) on cells treated with TMX (10 μM) by 78% (n=53), identified PKA in the acidification of lysosomes by SKF 81297. This was consistent with the ability of cell-permeant cAMP to acidify compromised lysosomes, and with the involvement of PKA in this general activation (Liu et al., supra, 2008).

In addition to its effects on tamoxifen treated cells, SKF 81297 was also effective at reversing the alkalinization produced by chloroquine, reducing lysosomal pH from 5.60±0.14 to 5.11±0.09 (n=24, p<0.005). However, SKF 81297 had no effect on the baseline lysosomal pH of cells that had been treated with neither tamoxifen nor chloroquine (n=10; p=0.99). The inability of SKF 81279 to decrease baseline lysosomal pH is consistent with data indicating cAMP exhorts an acidification of greater magnitude from cells with alkalized (“abnormal”) lysosomes than from baseline (“normal” acidic pH) (Liu et al., Amer. J. Physiol. Cell Physiol. 2012).

### Example 5

### Acidifying Effect of Single Dose of SKF 81297 is Sustained

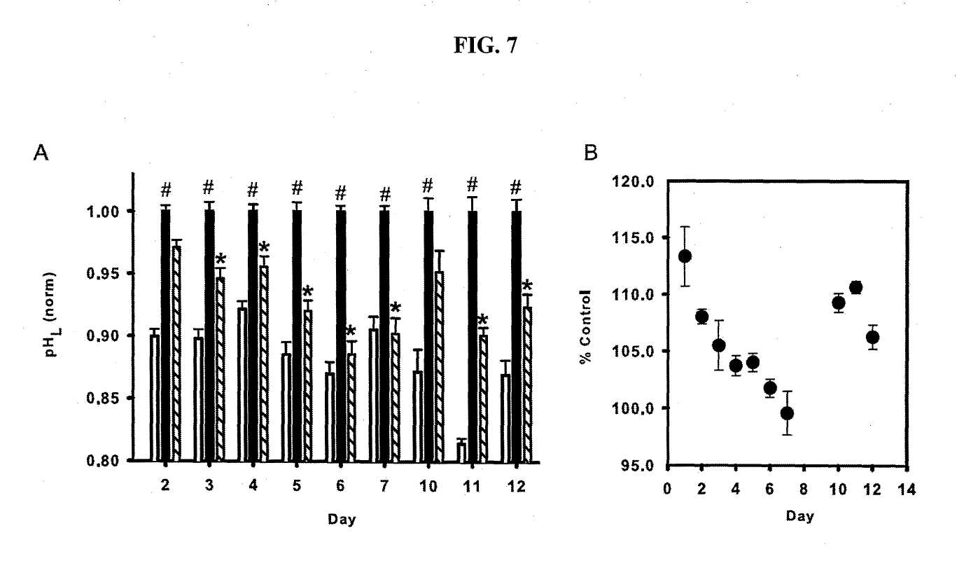

Although the experiments above have shown that stimulation of D1-like receptors restored the lysosomal pH in compromised RPE cells, they were all conducted over the course of several hours. To confirm that D1-like receptor stimulation induced a sustained restoration of lysosomal pH in compromised RPE cells, agonist SKF 81297 was added to chloroquine-treated cells, as chloroquine has been reported to induce prolonged effects in RPE cells in vivo (Peters et al., Opthamol. Res. 38:83-88 (2006)). Confluent cells were treated with 10 μM chloroquine in the presence and absence of SKF 81297 (10 μM) in the presense (hash bar on FIG. 7A) or absence (solid black bar on FIG. 7B) of 10 μM SKF 8129 day 0 in 2 to 5 trials, and the lysosomal pH was measured over at least the next 12 days, but the SKF 81297 was not refreshed after the initial treatment. The pH levels were normalized to the mean value in chloroquine for each day's measurements to compensate for variation across trials. # CHQ versus control, p<0.05, *p<0.05 SKF 81297 versus CHQ; n=16-40. Medium was not changed for control or treatment wells. Measurements were performed in the several trials, each measuring lysosomal pH on a different combination of days. While absolute levels varied somewhat based upon both the plating and the measurement day, trends were clearly evident. Extended exposure to 10 μM chloroquine induced a relatively constant elevation in lysosomal pH. In contrast, it was apparent that the acidifying effect of SKF 81297 changed with exposure duration (FIG. 7A).

SKF 81297 lowered pH_{L} more effectively with increased exposure time. Remarkably, exposure of compromised cells to SKF 81297 completely restored the lysosomal pH to baseline levels at day 7 (FIG. 7B). The effectiveness of a single dose of SKF 81297 peaked 7 days after treatment, producing a near-complete restoration of pH_{L} as calculated from the mean of the two to five trials derived from 16 to 40 measurements. Although the magnitude of the acidification was reduced, SKF 81297 still produced a significant acidification up to at least 12 days, the last day examined. The effect of treatment may have continued well past the end of the example at 12 days, but it was no longer measured. No difference between treated and control cells was discerned visually. Thus, a single dose of SKF 81297 produced a cumulative or sustained reacidification effect of the compromised cells.

This then provided a model where the cAMP increase following dopamine receptor stimulation by SKF 81297 affects the regulation of lysosomal pH, but does not alter its baseline maintenance. Thus, the selective activity of SKF 81297 on alkalized cells makes the treatment of impaired tissue ideally suited, as the lysosomal pH of any healthy cells appears to be minimally affected.

### Example 6

### Molecular Identification of D5 Receptor Subtype

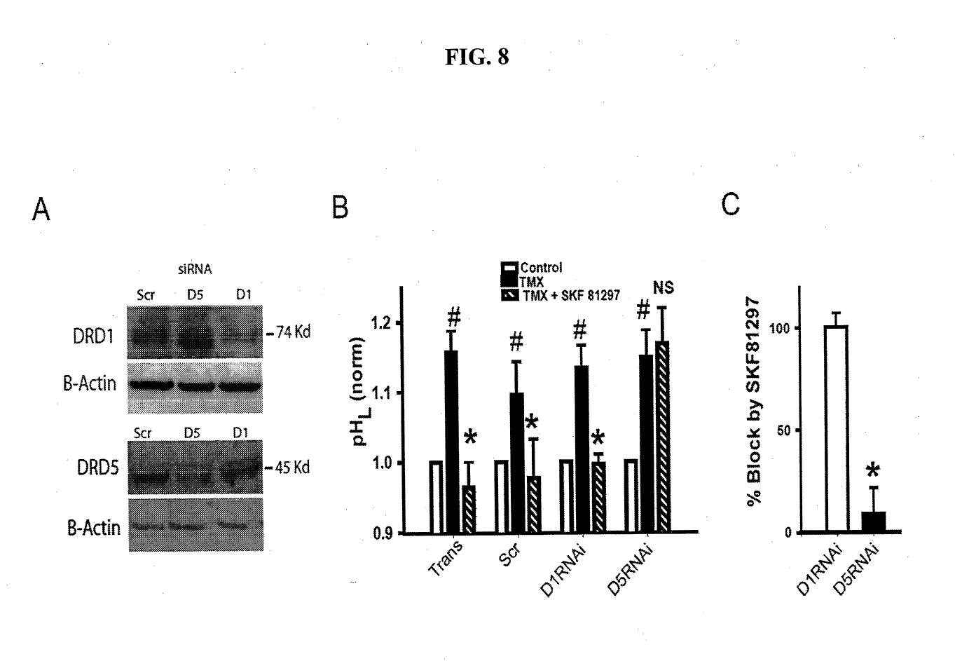

As available pharmacological tools are currently unable to distinguish between the D1 and D5 receptors with reasonable specificity, molecular approaches were used to determine which receptor was responsible for the lysosomal acidification, for example by agonist SKF 81297. Western blots confirmed that an antibody against the D1DR detected a band at expected size of 74 kD. The intensity of the band was reduced by siRNA against the D1DR (FIG. 8A) siRNA against the D5 receptor reduced expression of the D5DR, but not the D1DR. An antibody against the D5DR detected a band at the expected size of 45 kD, with the intensity of the band reduced by siRNA against the D5DR. When normalized to β-actin 72 hours post transfection and quantified to levels in scrambled siRNA (abbreviated “Scr” in FIG. 8), the band intensity of D1DR was decreased to 57% by siRNA against D1DR while siRNA against the D5DR increased expression to 162% of scrambled levels. Levels of D5DR were decreased by siRNA against D5DR to 75%, with siRNA against the D1DR leading to 106% of scrambled levels.

As these siRNA probes were able to selectively reduce expression of the receptor target protein, their effect on the ability of SKF 81297 to restore acidity was tested. The baseline pH did not differ between cells transfected with scrambled siRNA, D1DR siRNA, D5DR siRNA, or transcription controls in seven separate transfection experiments (p 0.74, 0.68 and 0.53 vs. scrambled, respectively; transfection itself had a slight alkalizing effect). To control for variations that occurred between trials, pH values were normalized to the mean value for scrambled control for each experiment, but still there was no difference in baseline levels (p>0.22). However, significant differences were observed when the ability of SKF 81297 to acidify the lysosomes of compromised cells was examined. Tamoxifen produced a similar alkalinization of lysosomes in all cells. SKF 81297 acidified the lysosomes of cells transfected with scrambled siRNA or exposed to transfection medium.

While SKF 81297 likewise acidified the lysosomes of cells transfected with D1DR siRNA, the drug had little effect on lysosomal pH in cells exposed to D5DR siRNA (FIG. 8B). Paired t-test #p<0.05, TMX versus Control; * p<0.05, TMX versus TMX+SKF 81297, n=6 plates, 2-4 wells each. Data were normalized to the mean control in each set to account for variation between each separate set of transfections.

When the % reacidification of the effect of tamoxifen was calculated, SKF 81297 blocked 100.4±9.1% of the alkalizing effects of tamoxifen in the presence of D1DR siRNA, while it blocked only 10.4±19.1% of the alkalinization in the presence of D5DR siRNA (p=0.006, FIG. 8C). This indicated that the response was mediated by the D5 dopamine receptor. In FIG. 8C, the magnitude of the acidification by 10 μM SKF 81297 was defined as percent reacidification=100 X (TMX−(TMX+SKF))/(TMX−Control). The percent reacidification was unaffected when cells were transfected with siRNA against D1DR. However, siRNA against the D5DR reduced the percent reacidification to only 10%, identifying the D5 receptor in the reacidification by SKF 81297. Paired t-test, *p=0.006 versus D1RNAi, n=4.

Of note, although the immunoblots suggest an increase in D1DR expression with D5DR siRNA knockdown, there was no evidence of an effect on a physiological level as baseline lysosomal pH did not differ significantly between cells treated with D1DR siRNA or D5DR siRNA, and as mentioned, the effect of SKF 81297 was decreased, not increased, by D5DR siRNA. This further supports the role for the D5DR in lysosomal acidification.

### Example 7

### D5 Stimulation Enhances Degradative Activity of RPE Lysosomes

Degradative lysosomal enzymes are pH sensitive, acting optimally over a relatively narrow range of acidic values. As such, conditions which elevate lysosomal pH are predicted to reduce rates of degradation, whereas treatments to reacidify lysosomes are predicted to enhance degradation. RPE lysosomes are required to degrade photoreceptor outer segments phagocytosed daily (Kevany et al., Physiology (Bethesda) 25:8-15 (2010)). As such, the effect of lysosomal pH manipulation of outer segment degradation was tested.

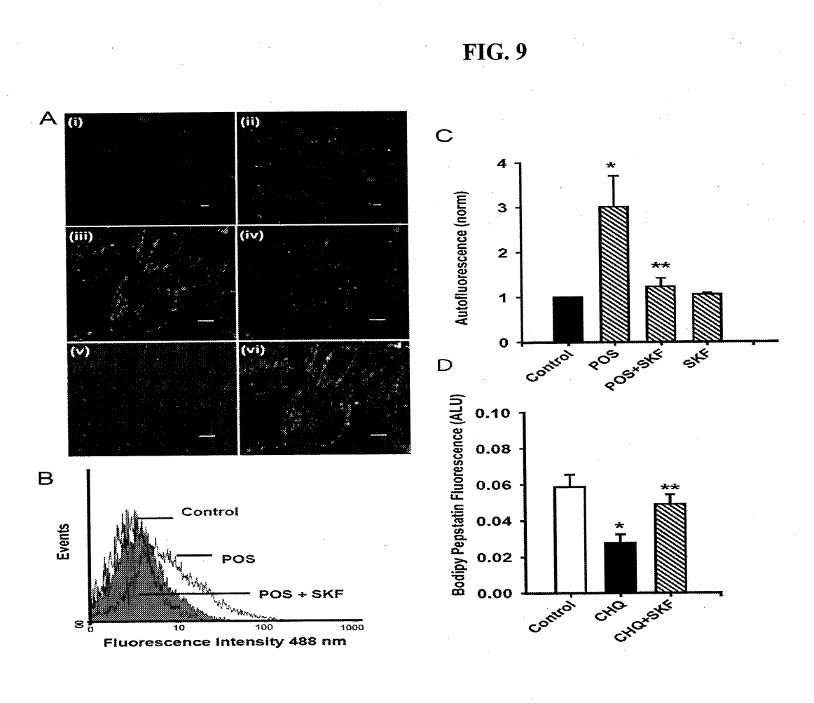

Initial experiments were designed to confirm that outer segments were internalized to the lysosomes. Unlabeled photoreceptor outer segments were fed to confluent ARPE-19 cells for 2 h and then medium was returned. This procedure was repeated every other day for 7 days. On the final day the cells were maintained in outer segment-free medium for 2 h to ensure sufficient time for binding, phagocytosis, and trafficking. While cells not exposed to POS displayed little autofluorescence, cells exposed to POS displayed clear spots of autofluorescence when excited at 488 nm (FIG. 9A i, ii). As this pattern of autofluorescence indicates organelle staining, costaining with LysoTracker Red was examined. The punctate pattern of autofluorescent staining from outer segments overlapped with the pattern for LysoTracker Red, indicating that most of the autofluorescence was restricted to lysosome-like organelles at this point (FIG. 9A iii-vi).

Having established that photoreceptor outer segments were delivered to lysosomes within 2 h, the autofluorescence was quantified and the ability of SKF 81297 to alter this autofluorescence was calculated. The cells were fed photoreceptor outer segments for 2 h, kept in outer segment-free medium for 2 h to allow for internalization. After 2 h, cells were fed 10 μM SKF 81297 for 19 h, at which point the outer segment feeding was resumed. This complex “pulse-chase” protocol was followed to ensure that drug treatment did not interfere with POS binding or internalization.

Treatment with photoreceptor outer segments substantially increased cellular autofluorescence, whereas treatment with SKF 81297 clearly decreased autofluorescence (FIG. 9B). Cells were fed POS for 3 h, washed, and 2-h chase period were allowed for outer segment delivery to the lysosomes. At this point, 10 μM SKF 81297 was added to the cells (adding the drug after the 2-h interval ensured effects were restricted to outer segment digestion and did not alter binding or phagocytosis). This two-stage treatment was repeated every 1-2 days for 1 week, with a total of three treatments. Cells were dissociated and the autofluorescence excited at 488 mu was determined using flow cytometry. Compared with control cells, exposure to POS shifted the fluorescence to the right (red), indicating an increased fluorescence. Treatment with SKF 81297 shifted the curve back to the left (greenward) as autofluorescence was reduced.

In five trials, treatment with outer segments raised autofluorescence over 3-fold, while exposure to SKF 81297 reduced autofluorescence by 73±12% (FIG. 9C). The mean autofluorescence was increased by incubation with POS, but restored low levels by treatment with a D5DR agonist, such as SKF 81297. SKF 81297 alone did not alter autofluorescence levels. Bars represent the mean±SEM fluorescence in each sample and are representative of results in three separate experiments. Data were normalized to peak levels in untreated cells to control for variation between trials. *p<0.05 versus control; **p<0.05 versus POS. This indicated that stimulation of the D5 receptor enhanced digestion of photoreceptor outer segments.

To provide additional evidence that stimulation of the D5 receptor increased lysosomal activity, the binding of the fluorescent Bodipy-pepstatin A to cells was assessed. Pepstatin A inhibits the lysosomal protease cathepsin D, and thus, fluorescence is indicative of cathepsin D activity in situ. Incubation of cells with 10 μM of chloroquine significantly decreased the fluorescence, as expected. However, coincubation of the D5DR agonist SKF 81297 with the chloroquine substantially increased the pepstatin A fluorescence (FIG. 9D). While binding of the probe was reduced by treating cells with 10 μM chloroquine for 48 h, concurrent exposure to 10 μM SKF 81297 restored fluorescence. These results are consistent with chloroquine decreasing activity of pH sensitive lysosomal enzyme cathepsin D, and of a D5DR agonist (SKF 81297) restoring enzyme activity. *p<0.05 vs. control; **p<0.05 CHQ vs. CHQ+SKF, n=13.

These results were consistent with the ability of lysosomal alkalinization by chloroquine to decrease the activity of cathepsin D, and the reacidification of lysosomes by action of a D5DR agonist, such as SKF 81297, to restore activity. Together with the ability of a D5DR agonist to stimulate the dopamine receptors to reduce the autofluorescence associated with photoreceptor outer segments, these findings demonstrate that agonist stimulation of the D5 receptor increased the activity of degradative lysosomal enzymes in compromised cells.

### Example 8

### Stimulation of D5 Receptors Acidifies Lysosomes from RPE Cells of ABCA4^{-/-} Mice

This experiment examined the effect of direct challenge of RPE cells by exposure to N-retinylidene-N-retinylethanolamine (A2E). A2E is known to elevate the lysosomal pH of cultured RPE cells (See, Holz et al., supra, 1999; Liu et al., supra, 2008). The ABCA4^{-/-} mouse model of recessive Stargardt's disease is, of course, characterized by excessive accumulation of A2E (Mata et al., supra, 2001); and the lysosomal pH of these mice is elevated as compared with age-matched controls (Liu et al., supra, 2008). Given the potential importance for RPE pathophysiology, the ability of D5 receptor stimulation to lower lysosomal pH in RPE cells from ABCA4^{-/-} mice was examined.

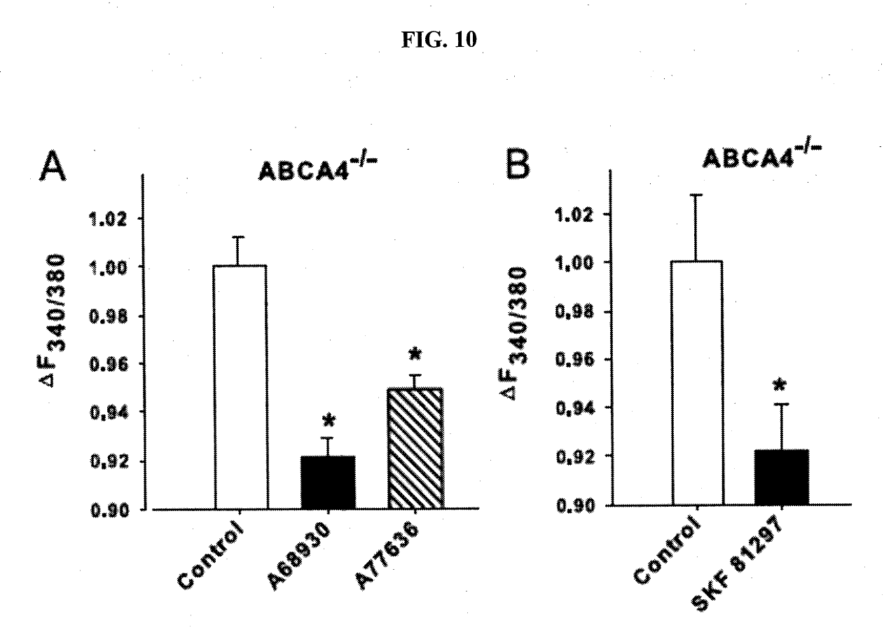

RPE cells were freshly isolated from ABCA4^{-/-} mice and lysosomal pH was measured in vitro. Exposure of RPE cells from 11-month-old mice to 1 μM A68930 or 1 μM A77636 decreased lysosomal pH (FIG. 10A). Interestingly, these drugs had no significant effect on the lysosomal pH of 5-month-old ABCA4^{-/-} mice. Additional experiments demonstrated that SKF 81297 (50 μM) also reduced the signal from 12-month-old ABCA4^{-/-} mice (FIG. 10B).

The ability of D5 receptor stimulation to enhance outer segment degradation in RPE cells with compromised (alkalized) lysosomes has implications for patients with macular degenerations, such as Stargardt's disease, because the lysosomal pH was increased in RPE cells from the ABCA4^{-/-} mouse model of the disease (Liu et al., supra, 2008). As such, the ability of receptor agonists to acidify lysosomes from RPE cells taken from older ABCA4^{-/-} mice is important, for it implies that the mechanisms necessary to mediate receptor-driven reacidification of lysosomes are still functioning even though the lysosomes in the cells have been distressed for an extended period. The lysosomal pH increased with age in these mice (Liu et al. supra, 2008), consistent with the enhanced accumulation of A2E with age (Mata et al., supra, 2000). The negligible effect of D5DR agonists in younger mice with near-normal lysosomal pH appears to be related to the increased magnitude of acidification induced by cAMP when given to cells with alkalized lysosomes.

### Example 9

### Effect of D5DR Agonist on Extracellular IL-6 and Cytoplasmic Ca²⁺ Release in Cells with Higher (More Alkaline) pH

Recent evidence suggests that lysosomes are a storage site of Ca²⁺ and inflammatory cytokines. Elevation (meaning greater alkalization) of lysosomal pH (pH_{L}), for instance in compromised RPE cells, thus leads to the release of calcium and inflammatory cytokines, such as IL-6. Accordingly, the effect of the ability of D1/D5 agonist receptor agonists, such as SKF81297, to reacidify the lysosomes and restore lysosomal enzyme activity was examined to determine if the agonists also reduced the release of calcium and pro-inflammatory cytokines.

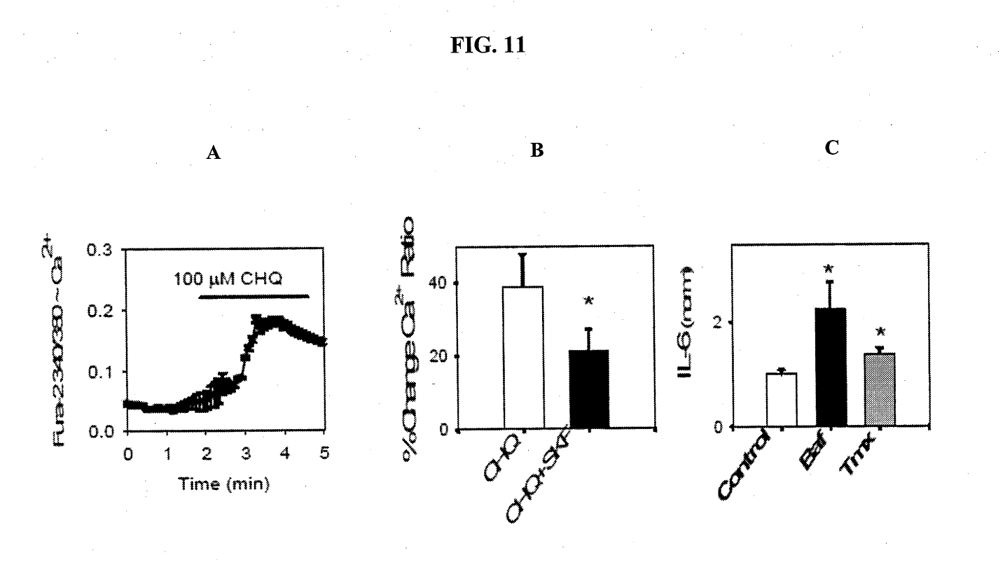

As shown in FIGS. 11A-11C, measurement of intracellular calcium with the indicator fura-2 confirmed that raising lysosomal pH (increasing alkalization) with chloroquine led to the release of Ca²⁺ into the cells. Howver, this chloroquine-dependent release of calcium was attenuated by administering 10 μM SKF 81297 (n=12). Similarly, raising lysosomal pH with bafilomycin or tamoxifen caused a release of cytokine IL-6 into the extracellular bath (n=9). *p<0.05, which was also attenuated by administration of a D5DR agonist (SKF 81297).

As a result, raising lysosomal pH causes release of extracellular IL-6 and cytoplasmic Ca²⁺, but administration of a D5DR (SKF81297) to stimulate the dopamine receptors and thereby reduce (acidify) pH_{L} of the compromised cells blocks and prevents release of the extracellular IL-6 and cytoplasmic Ca²⁺. Thus, it is shown for the first time that administering a D1/D5 agonist receptor agonist, such as SKF 81297, to reacidify the lysosomes of compromised RPE cells, reduces the release of calcium and pro-inflammatory cytokines in accordance with its reduction (acidification) of pH_{L}.

In sum, since phagocytosis generally follows a circadian pattern, temporal control of the delivery of agonists appear to enable the effects on phagocytosis and lysosomal degradation to be separated in vivo as they were in vitro. In this regard, chronic treatment with 1-methyl-4-phenyl-1,2,3,6- tetrahydropyridine decreased the dopaminergic amacrine cells in the retina and significantly increased the number of highly fluorescent yellow lipofuscin granules in the RPE (Mariani et al., Neurosci. Lett. 72:221-226 (1986)). The lipofuscin associated with dopamine reduction displayed the same spectral profile as the lipofuscin in RPE cells from older animals. As a result, this provides a model whereby dopamine released from the amacrine cells normally keeps the lysosomal pH (pH_{L}) of RPE cells low and outer segment degradation running smoothly; but the removal of this source of dopamine appears to lead to lysosomal alkalization and accumulation of autofluorescent debris.

Conversely, such a model is consistent with the results in FIG. 9, whereby application of D5DR agonist, exemplified by SKF 81297 substantially reduced the degree of autofluorescence in RPE cells. The activity of a single dose of SKF 81297 peaked 7 days after treatment, producing a near-complete restoration of pH_{L} as calculated from the mean of the two to five trials, providing from 16 to 40 measurements. Moreover, although the magnitude of the acidification was reduced over time, SKF 81297 still produced a significant acidification up to the last day of examination at day 12. Yet, there was no visible physical difference between the treated and the control cells. Accordingly, a single dose of a D5DR agonist produced a continuous and cumulative reacidification of alkalized, i.e., compromised cells. This provided a model where the cAMP increase following dopamine receptor stimulation by a D5DR agonist modulated the regulation of lysosomal pH, but it did not alter its baseline maintenance. Thus, the selective activity of a D5DR agonist on alkalized cells makes the treatment of impaired tissue ideal, since the lysosomal pH of any healthy cells appears to be minimally affected. Overall, these findings demonstrate that D5 receptor stimulation is a critical pathway to enhance degradation in RPE cells in vivo.

Administration of a D5DR agonist (exemplified by SKF 81297) increased the activity of degradative lysosomal enzymes in compromised cells, and the degradation of ingested photoreceptor outer segments by RPE cells was also increased by stimulation of D5 dopamine receptors. D1/D5 receptor agonists reacidified lysosomes in cells alkalized by chloroquine or tamoxifen, with acidification dependent on protein kinase A. Knockdown with siRNA confirmed acidification was mediated by the D5 receptor. Exposure of RPE cells to outer segments increased lipofuscin-like autofluorescence, but treatment with a D5DR agonist reduced autofluorescence. Likewise, exposure to a D5DR agonist increased the activity of lysosomal protease cathepsin D in situ. D5DR stimulation also acidified lysosomes of RPE cells from elderly ABCA4^{-/-} mice, a model of recessive Stargardt's retinal degeneration. Thus, methods are provided in the present invention for slowing the progression of AMD by restoring an optimal acidic pH_{L} to compromised lysosomes in the RPE cells, and an effective treatment is provided for reversing macular degeneration and the damaging effects of abnormally elevated pH_{L}, particularly as found in AMD and in Stargardt's disease.

The disclosure of each patent, patent application and publication cited or described in this document is hereby incorporated herein by reference, in its entirety.

While the foregoing specification has been described with regard to certain preferred embodiments, and many details have been set forth for the purpose of illustration, it will be apparent to those skilled in the art without departing from the spirit and scope of the invention, that the invention may be subject to various modifications and additional embodiments, and that certain of the details described herein can be varied considerably without departing from the basic principles of the invention. Such modifications and additional embodiments are also intended to fall within the scope of the appended claims.

What is claimed is:

1. A method of treating age-related macular degeneration (AMD) or Stargardt's disease in a patient subject to, or symptomatic thereof, the method comprising exogenously administering or up-regulating expression of a D1/D5 dopamine receptor agonist to compromised retinal pigment epithelium (RPE) cells of the patient's eye; stimulating D1-like dopamine receptors therein; and thereby restoring normal lysosomal pH (pH_{L}), or reacidifying abnormally elevated pH_{L}, in the RPE cells.

2. The method of claim 1, further comprising elevating cAMP by administering or stimulating receptors coupled to a Gs protein in an amount sufficient to decrease the elevated pH_{L} or restore acidity of lysosomes in the RPE cells.

3. The method of claim 1, wherein the family of D1-like dopamine receptors comprises D1 dopamine receptor (D1DR) and D5 dopamine receptor (D5DR).

4. The method of claim 3, wherein administering D5 dopamine receptor (D5DR) agonists, selected from the group consisting of A68930; A77636, and SKF 81287, effects increasing lysosomal activity, causing reacidification of lysosomal pH (pH_{L}) in aged or alkalized RPE cells having D5 receptors.

5. The method of claim 4, wherein stimulating the D5 receptor (D5DR) further effects greater increasing of lysosomal activity and greater decreasing of pH_{L} in the RPE cells, as compared to the effect of stimulating the D1 dopamine receptors.

6. The method of claim 5, wherein stimulating the D5 receptor (D5DR) further effects enhancing digestion of photoreceptor outer segments of the RPE cells.

7. The method of claim 5, wherein stimulating the D5 receptor (D5DR) further effects decreasing of accumulated autofluorescent photoreceptor debris in the RPE cells.

8. The method of claim 5, wherein administering SKF 81297 as a D5 dopamine receptor (D5DR) agonist effects increasing lysosomal activity, causing reacidification of lysosomal pH (pH_{L}) in compromised, aged or alkalized RPE cells.

9. The method of claim 8, wherein stimulating D5DR of compromised, ages or alkalized RPE cells by administering SKF 81297 agonist effects regulating lysosomal pH (pH_{L}), without altering baseline maintenance.

10. The method of claim 9, wherein stimulating D5DR of compromised RPE cells by administering a single dose of SKF 81297 agonist on day 0, effects increasing activity of degradative lysosomal enzymes and restoring pH_{L} in the compromised cells over a sustained and continuous time for at least 12 days.

11. A method of using a D5DR agonist to stimulate D5DR in compromised, aged or alkalized retinal pigment epithelium (RPE) cells, the method comprising exogenously administering the D5DR agonist to the compromised RPE cells; stimulating D5 dopamine receptor activity in the RPE cells; thereby regulating and restoring normal lysosomal pH (pH_{L}), or reacidifying abnormally elevated pH_{L}, in the cells without altering baseline maintenance.

12. The method of claim 11, wherein the D5 dopamine receptor (D5DR) agonist is selected from the group consisting of A68930; A77636, and SKF 81287.

13. The method of claim 12, further comprising enhancing digestion of photoreceptor outer segments of the RPE cells.

14. The method of claim 12, further comprising decreasing of accumulated autofluorescent photoreceptor debris in the RPE cells.

15. The method of claim 12, wherein administering a single dose of SKF 81297 agonist on day 0, further effects increasing activity of degradative lysosomal enzymes and restoring pH_{L} in the compromised cells over a sustained and continuous time for at least 12 days.

16. The method of claim 11, wherein the retinal pigment epithelium (RPE) cells are those of a patient subject to, or symptomatic of age-related macular degeneration (AMD) or Stargardt's disease.

17. A method of restoring photoreceptors to the eye of a patient subject to, or symptomatic of, reduced photoreceptor activity or lipofuscin accumulation in RPE cells, the method comprising acidifying or restoring lysosomal pH (pH_{L}) in compromised RPE cells through a D5 dopamine receptor (D5DR)-mediated pathway, thereby restoring degradation and removal of phagocytosed photoreceptor outer segments, and enzymatically decreasing or blocking damaging accumulations of lipofuscin and metabolic waste in the RPE cells before debris accumulates, permitting repopulation of the photoreceptors.

18. The method of restoring photoreceptors of claim 17, the method comprising exogenously administering or up-regulating expression of a D1/D5 dopamine receptor agonist in or to the RPE cells; stimulating D1-like dopamine receptors; thereby restoring degradation and removal of phagocytosed photoreceptor outer segments, and enzymatically decreasing or blocking damaging accumulations of lipofuscin and metabolic waste in the RPE cells before debris accumulates, permitting repopulation of the photoreceptors.

19. The method of restoring photoreceptors of claim 18, wherein a D5 dopamine receptor (D5DR) agonist is selected from the group consisting of A68930; A77636, and SKF 81287.

20. The method of claim 19, comprising administering SKF 81297 agonist for stimulating D5DR activity of compromised RPE cells, thereby regulating and reacidifying lysosomal pH (pH_{L}), without altering baseline maintenance.

21. A method for reducing or blocking release of extracellular proinflamatory cytokines and/or cytoplasmic Ca²⁺ associated with elevated (more alkaline) pH_{L} of compromised RPE cells, the method comprising exogenously administering or up-regulating expression of a D1/D5 dopamine receptor agonist to the compromised RPE cells; stimulating D1-like dopamine receptors therein and thereby restoring normal lysosomal pH (pH_{L}), or reacidifying abnormally elevated pH_{L}, and blocking or preventing release of the extracellular pronflammatory cytokines and the cytoplasmic Ca²⁺ as a result of acidification of the pH_{L}.
